(19) United States
(12) Patent Application Publication
(10) Pub. No.: US 2026/0209715 A1
(43) Pub. Date: Jul. 23, 2026

(54) CGMP COMPLIANT DIFFERENTIATION OF IPSCS TO IECS

(71) Applicant: Cedars-Sinai Medical Center, Los Angeles, CA (US)

(72) Inventors: Dhruv Sareen, Porter Ranch, CA (US); Arwin Aghamaleky-Sarvestany, Los Angeles, CA (US); Sarah Parker, Los Angeles, CA (US); Nethika Ariyasinghe, Los Angeles, CA (US)

(73) Assignee: Cedars-Sinai Medical Center, Los Angeles, CA (US)

(21) Appl. No.: 19/133,568

(22) PCT Filed: Dec. 8, 2023
(86) PCT No.: PCT/US2023/083118

§ 371 Date: May 28, 2025

Related U.S. Application Data
(60) Provisional application No. 63/431,287, filed on Dec. 8, 2022.

(51) Int. Cl. C12N 5/071; A61K 35/44

(52) U.S. Cl. CPC C12N 5/069; A61K 35/44; C12N 2500/02; C12N 2501/115; C12N 2501/155; C12N 2501/165; C12N 2503/02; C12N 2506/45; C12N 2533/52

(57) ABSTRACT

Described herein are methods for current good manufacturing practices (cGMP) compliant differentiation of induced pluripotent stem cells into endothelial cells (iECs), iECs made by these methods, and methods of using these iECs.

### CROSS-REFERENCE TO RELATED APPLICATIONS

This application includes a claim of priority under 35 U.S.C. § 119(e) to U.S. provisional patent application No. 63/431,287, filed Dec. 8, 2022, the entirety of which is hereby incorporated by reference.

### STATEMENT REGARDING FEDERALLY SPONSORED RESEARCH OR DEVELOPMENT

This invention was made with Government support under W81XWH-20-9-0022 awarded by Department of Defense. The Government has certain rights in the invention.

### FIELD OF INVENTION

This invention relates to cGMP-grade iPSC-derived endothelial cells, manufacturing and uses thereof.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Vascular endothelial cells, which line the luminal surface of vessels are essential components in organ regeneration and wound healing. Recent advances in the field of induced pluripotent stem cells (iPSCs) research have opened a new avenue for generation of vascular cells. The ability of human iPSCs to self-renew indefinitely and differentiate into various types of cells afford us with an unprecedented opportunity to reconstruct and replace damaged tissues. Among major applications of vascular stem cell therapy could be treatment of severe combat injuries in military service members.

Several methods have been developed to differentiate human induced endothelial cells (iECs) from hiPSCs. However, widespread implementation of iPSC-based therapies is limited by lack of standardized manufacturing processes and quality control assays, insufficient number of cells, immunogenicity of the allogeneic hPSC-derived cells and inefficient differentiation protocols. As such, there remains a need in the art for a consistent and highly efficient GMP-compatible method to generate functional human iPSCs-derived endothelial cells.

### SUMMARY OF THE INVENTION

The following embodiments and aspects thereof are described and illustrated in conjunction with compositions and methods which are meant to be exemplary and illustrative, not limiting in scope.

Various embodiments provide for a method of generating endothelial cells from induced pluripotent stem cells (iECs), comprising
(i) differentiating induced pluripotent stem cells (iPSCs) into mesoderm cells by: removing cGMP feeder-free iPSC maintenance medium from plated induced pluripotent stem cells (iPSCs) and adding mesoderm induction medium, and culturing the iPSCs for about 1.5-2.5 days in about 36-38 C and in the presence of about 4-6% CO₂ to generate mesoderm cells;
(ii) differentiating the mesoderm cells into vascular progenitor cells by: removing the mesoderm induction medium and adding vascular progenitor medium, and culturing the cells for about 1.5-2.5 days in about 36-38 C and in the presence of about 4-6% CO₂ to generate vascular progenitor cells;
(iii) differentiating the vascular progenitor cells into iECs by: dissociating the vascular progenitor cells by using a first dissociation enzyme for about 4-6 minutes at 36-38 C and in the presence of about 4-6% CO₂, wherein the dissociation enzyme comprises an animal origin free enzyme that cleaves peptide bonds on the C-terminal sides of lysine and arginine; neutralizing the vascular progenitor cells by using basal endothelial cell growth medium MV2 plus about 9-11% human AB serum, spinning the vascular progenitor cell solution, removing the basal endothelial medium, and resuspending the vascular progenitor cells in GMP-grade iEC differentiation medium, wherein the human AB serum comprises human serum lacking antibodies against A and B blood-type antigens that was converted from Pooled Plasma (Human), Solvent/Detergent Treated; plating the vascular progenitor cells at about 9,000-11,000 cells/cm² with iEC differentiation medium onto a dish that was previously coated with 4-6 ug/ml human recombinant laminin 421 in PBS Mg+/Ca+ for about 1.5-2.5 hours at about 36-38 C and in the presence of about 4-6% CO₂, culturing the vascular progenitor cells at about 36-38 C and in the presence of about 4-6% CO₂ for about 6-8 days, feeding about every other day with iEC differentiation medium to generate iECs;
(iv) further culturing the iECs by: dissociating the iECs by using animal origin free enzyme that cleaves peptide bonds on the C-terminal sides of lysine and arginine (1×) for about 4-6 minutes at about 36-38 C and in the presence of about 4-6% CO₂; neutralizing the iECs using by using basal endothelial cell growth medium MV2 plus 9-11% Human AB serum, spinning the iEC solution, removing the basal endothelial medium, and resuspending the iECs in GMP-grade iEC differentiation medium, wherein the human AB serum comprises human serum lacking antibodies against A and B blood-type antigens that was converted from Pooled Plasma (Human), Solvent/Detergent Treated; plating the iECs at about 9,000-11,000 cells/cm² with iEC differentiation medium onto a dish that was previously coated with 5 ug/ml human recombinant laminin 421 in PBS Mg+/Ca+ for about 1.5-2.5 hours at about 36-38 C and in the presence of about 4-6% CO₂, culturing the iECs at about 36-38 C and in the presence of about 4-6% CO₂ for about 9-11 days, feeding about every other day with iEC medium, thereby generating iECs expressing CD31 and CD144.

In various embodiments, the method can further comprise dissociating the iECs that are generated in step (iv).

In various embodiments, the method can further comprise providing plated iPSCs, wherein the plated iPSCs are made by: dissociating iPSCs with a cGMP enzyme-free human pluripotent stem cell selection and passaging reagent for 5 minutes and neutralizing the iPSCs with iPSC maintenance medium; triturating the iPSCs about 10-15 times to break large colonies of iPSCs to smaller colonies of iPSCs and to have a homogenous cell population that is about 60-200 um diameter per iPSC colony; dissociating a subset of colony solution as single cells by using a dissociation solution, for 5 minutes at 37 C water bath, wherein the dissociation solution comprises a cell detachment solution of proteolytic and collagenolytic enzymes and does not contain mammalian or bacterial derived products; neutralizing the dissociation solution by using a cGMP feeder-free iPSC maintenance medium, spinning the cell solution, removing the medium and resuspending the iPSCs in iPSC maintenance medium; removing laminin solution from a plating dish that previously coated with 5 ug/ml human recombinant laminin 521 in PBS Mg+/Ca+ for 2 hours in 37 C and in the presence of 5% CO₂; adding cGMP feeder-free iPSC maintenance medium to the plating dish; adding about 2000 iPSCs/cm² to the plating dish and culturing at 37 C and in the presence of 5% CO₂; changing the cGMP feeder-free iPSC maintenance medium daily for an additional 2 days.

In various embodiments, the mesoderm induction medium can comprise a final concentration of about 5-7 uM CHIR99021 and about 1:900-1:1100 Antibiotic-Antimycotic (PSA) in iPSC differentiation medium, wherein the iPSC differentiation medium comprises a defined, animal component-free medium for differentiation of human ES and iPS cells to multiple lineages.

In various embodiments, the vascular progenitor medium can comprise final concentrations of about 20-30 ng/ml Bone Morphogenetic Protein 4 (BMP4), about 8-12 ng/ml FGF2, about 40-60 ng/ml VEGF165 and about 1:900-1:1100 Antibiotic-Antimycotic (PSA) in iPSC differentiation medium.

In various embodiments, the iEC differentiation medium can comprise Basal MV2, PSA, Human AB Serum (AB serum), Epidermal Growth Factor (EGF), Basic Fibroblast Growth Factor (FGF2), Long R3 Insulin-like Growth Factor (IGF-1), Vascular Endothelial Growth Factor 165 (VEGF 165), Ascorbic acid, and Hydrocortisone.

In various embodiments, the iEC differentiation medium can comprise Basal MV2, 1:900-1:1100 PSA, 0.05-0.15 ml/ml Human AB Serum (AB serum), 4-6 ng/ml Epidermal Growth Factor (EGF), 8-12 ng/ml Basic Fibroblast Growth Factor (FGF2), 18-22 ng/ml Long R3 Insulin-like Growth Factor (IGF-1), 240-280 ng/ml Vascular Endothelial Growth Factor 165 (VEGF 165), 0.5-1.5 ug/ml Ascorbic acid, and 0.1-0.3 ug/ml Hydrocortisone.

In various embodiments, the method comprise
(i) differentiating induced pluripotent stem cells (iPSCs) into mesoderm cells by: removing cGMP feeder-free iPSC maintenance medium from plated induced pluripotent stem cells (iPSCs) and adding mesoderm induction medium, and culturing the iPSCs for about 2 days in about 37 C and in the presence of about 5% CO₂ to generate mesoderm cells;
(ii) differentiating the mesoderm cells into vascular progenitor cells by: removing the mesoderm induction medium and adding vascular progenitor medium, and culturing the cells for about 2 days in about 37 C and in the presence of about 5% CO₂ to generate vascular progenitor cells;
(iii) differentiating the vascular progenitor cells into iECs by: dissociating the vascular progenitor cells by using a first dissociation enzyme for about 5 minutes at 37 C and in the presence of about 5% CO₂, wherein the dissociation enzyme comprises an animal origin free enzyme that cleaves peptide bonds on the C-terminal sides of lysine and arginine; neutralizing the vascular progenitor cells by using basal endothelial cell growth medium MV2 plus about 10% human AB serum, spinning the vascular progenitor cell solution, removing the basal endothelial medium, and resuspending the vascular progenitor cells in GMP-grade iEC differentiation medium, wherein the human AB serum comprises human serum lacking antibodies against A and B blood-type antigens that was converted from Pooled Plasma (Human), Solvent/Detergent Treated; plating the vascular progenitor cells at about 10,000 cells/cm² with iEC differentiation medium onto a dish that was previously coated with Sug/ml human recombinant laminin 421 in PBS Mg+/Ca+ for about 2 hours at about 37 C and in the presence of about 5% CO₂, culturing the vascular progenitor cells at about 37 C and in the presence of about 5% CO₂ for about 7 days, feeding about every other day with iEC differentiation medium to generate iECs;
(iv) further culturing the iECs by: dissociating the iECs by using animal origin free enzyme that cleaves peptide bonds on the C-terminal sides of lysine and arginine (1×) for about 5 minutes at about 37 C and in the presence of about 5% CO₂; neutralizing the iECs using by using basal endothelial cell growth medium MV2 plus 10% Human AB serum, spinning the iEC solution, removing the basal endothelial medium, and resuspending the iECs in GMP-grade iEC differentiation medium, wherein the human AB serum comprises human serum lacking antibodies against A and B blood-type antigens that was converted from Pooled Plasma (Human), Solvent/Detergent Treated; plating the iECs at about 10,000 cells/cm² with iEC differentiation medium onto a dish that was previously coated with 5 ug/ml human recombinant laminin 421 in PBS Mg+/Ca+ for about 2 hours at about 37 C and in the presence of about 5% CO₂, culturing the iECs at about 37 C and in the presence of about 5% CO₂ for about 10 days, feeding about every other day with iEC medium, thereby generating iECs expressing CD31 and CD144.

In various embodiments, the method can further comprise dissociating the iECs that are generated in step (iv).

In various embodiments, the method can further comprise providing plated iPSCs, wherein the plated iPSCs are made by: dissociating iPSCs with a cGMP enzyme-free human pluripotent stem cell selection and passaging reagent for 5 minutes and neutralizing the iPSCs with iPSC maintenance medium; triturating the iPSCs about 10-15 times to break large colonies of iPSCs to smaller colonies of iPSCs and to have a homogenous cell population that is about 60-200 um diameter per iPSC colony; dissociating a subset of colony solution as single cells by using a dissociation solution, for 5 minutes at 37 C water bath, wherein the dissociation solution comprises a cell detachment solution of proteolytic and collagenolytic enzymes and does not contain mammalian or bacterial derived products; neutralizing the dissociation solution by using a cGMP feeder-free iPSC maintenance medium, spinning the cell solution, removing the medium and resuspending the iPSCs in iPSC maintenance medium; removing laminin solution from a plating dish that previously coated with Sug/ml human recombinant laminin 521 in PBS Mg+/Ca+ for 2 hours in 37 C and in the presence of 5% CO₂; adding cGMP feeder-free iPSC maintenance medium to the plating dish; adding about 2000 iPSCs/cm² to the plating dish and culturing at 37 C and in the presence of 5% CO₂; changing the cGMP feeder-free iPSC maintenance medium daily for an additional 2 days.

In various embodiments, the mesoderm induction medium can comprise a final concentration of about 6 μM CHIR99021 and about 1:1000 Antibiotic-Antimycotic (PSA) in iPSC differentiation medium, wherein the iPSC differentiation medium comprises a defined, animal component-free medium for differentiation of human ES and iPS cells to multiple lineages.

In various embodiments, the vascular progenitor medium can comprise final concentrations of about 25 ng/ml Bone Morphogenetic Protein 4 (BMP4), about 10 ng/ml FGF2, about 50 ng/ml VEGF165 and about 1:1000 Antibiotic-Antimycotic (PSA) in iPSC differentiation medium.

In various embodiments, the iEC differentiation medium can comprise Basal MV2, PSA, Human AB Serum (AB serum), Epidermal Growth Factor (EGF), Basic Fibroblast Growth Factor (FGF2), Long R3 Insulin-like Growth Factor (IGF-1), Vascular Endothelial Growth Factor 165 (VEGF 165), Ascorbic acid, and Hydrocortisone.

In various embodiments, the iEC differentiation medium can comprise Basal MV2, 1:1000 PSA, 0.1 ml/ml Human AB Serum (AB serum), 5 ng/ml Epidermal Growth Factor (EGF), 10 ng/ml Basic Fibroblast Growth Factor (FGF2), 20 ng/ml Long R3 Insulin-like Growth Factor (IGF-1), 260 ng/ml Vascular Endothelial Growth Factor 165 (VEGF 165), 1 ug/ml Ascorbic acid, and 0.2 ug/ml Hydrocortisone.

Various embodiments provide for a quantity of iECs made by any one method of the invention as described herein. In various embodiments, the quantity is a clinically meaningful quantity.

Various embodiments provide for a method of treating a condition in a subject in need thereof, comprising administering iECs made by the method of any one of claims 1-14 to the subject in need thereof.

In various embodiments, the condition can comprise transplantation, tissue reconstruction, ischemic trauma or injury, diabetes, cancer, or cosmetic condition.

In various embodiments, treating a condition can comprise treating a wound, treating an amputation, treating a damaged organ, or treating necrosed tissue.

Various embodiments provide for a method of drug screening, comprising: contacting a test drug to a population of iECs made by any one of the methods of the present invention described herein; measuring one or more parameters of the iECs; and identifying the drug as a candidate based on the measured one or more parameters.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels A-F) shows the extensive proteomic analysis reveals similarities between iECs and a human vascular EC line (HUVECs) protein expression. A) Principal Component Analysis (PCA) of iECs Day 21 generated from 3 different iPSC lines (EDi028-A, EDi042-A, and 03n14), HUVECs and iPSCs (EDi028-A, EDi042-A, and 03n14). B) Differentially expressed proteins Heat map showing the relative expression levels of proteins differentially expressed with adjusted p-values less than 0.01. C) Venn diagram showing the number of proteins which expression is shared by all 3 types of cells, or between iECs and iPSCs only, iECs and HUVECs only, or iPSCs and HUVECs only. D) Volcano plot of the log 10 adjusted p-value of each expressed protein on iPSCs vs HUVECs+iECs, or on iECs vs HUVECs. Transcripts that did not demonstrate differential expression with an adjusted p-value of less than 0.05 and a log 2 fold-change in either direction greater than 0.5 are plotted in grey. E,F) Enriched pathways seen in enriched iECs only vs seen in enriched HUVECs only.

FIG. 2 shows differential expression of proteins in endothelial cell types (HUVECs and iECs) vs. iPSCs. This demonstrated differences in functional pathways between the cell types. (Top) Volcano plot of the log 10 adjusted p-value of each expressed protein on iECs vs HUVECs. Negative values (blue) are higher in iECs while positive values (orange) are higher in HUVECs. EMT and glycolysis pathways were upregulated in iECs (bottom left), while IFN-gamma and oxidative phosphorylation were upregulated in HUVECs (bottom right).

FIG. 3 shows that post-translational modifications (PTMs) analysis reveals more shared PTMs between iECs and HUVECS and fewer between endothelial cell types and iPSCs. Phosphorylation, R and K methylation, K and n-term acetylation sites in HUVECs, iECs, and iPSCs were compared.

FIG. 4 (panels A-B) shows dual phosphorylation and lysine monomethylation site on AKT2 were elevated in HUVECS as compared to iECs. A) Intensity peaks demonstrate phosphorylation and lysine monomethylation on AKT2. B) Levels of the modified AKT2 site was elevated in HUVECS as compared to iECs while iECs contained the largest total amount of AKT2.

FIG. 5 (panels A-B) shows lysine monomethylation site on CTNNB1 was only present in endothelial cell types (iECs and HUVECs). A) Intensity peaks demonstrate lysine monomethylation of CTNNB1. B) Lysine methylation of CTNNB1 was present primarily in endothelial cell types (iECs and HUVECS) while KDM2A and KDM2B were present in higher levels in iPSCs.

FIG. 6A depicts a schematic of iECs differentiation using 5 and 10 K/cm² single iPS cell densities. FIG. 6B depicts protein expression of vascular EC markers CD144/CD31 at Days 11 and 21 using 5 and 10 K/cm2 single iPS as starting cell densities. FIG. 6C depicts statistical analysis of number of CD31/CD144 positive iEC cells between 5 and 10 K/cm² iPSC seeding cell densities.

FIG. 7A-7C depicts seeding iPS cell density for iEC differentiation. Flow analysis for co-expression of endothelial markers CD31/CD144 was performed in 3 different iPSC lines. Our results show seeding 5 K/cm² iPSCs induce low differentiation efficiency in 2 out of 3 iPSC lines on day 11 of differentiation. Differentiation efficiency between 1 and 2 K/cm² iPSCs are similar. We choose 2 k/cm² iPSC cell density for future experiments.

FIG. 8A-8G show development of GMP-compatible coating substrate.

FIG. 9A-9H depicts development of xeno-free iEC differentiation medium in 3 different iPSC lines. Flow analysis for co-expression of endothelial markers CD31/CD144 was performed in 3 different iPSC lines. Our results show Plastem in some cell lines induce low iEC differentiation and will not be used for future experiments. FIG. 9D-9F shows Representative Immunocytochemistry images of endothelial markers CD31, VEGFR2, CD144, VWF, proliferation marker Ki67 and smooth muscle marker SM22 at day 21 post differentiation. iECs were differentiated on laminin 421 from vascular progenitors using basal GMPLV2 medium that was supplemented by either FCS or AB serum or Plastem or HPL. As shown in these pictures Plastem induce low levels of endothelial marker expression and would not be used for future experiments. HPL also will not be used for future experiments because it is human derived but not GMP product. CD31 (Red), VEGFR2, CD144, VWF, ki67 and SM22 (green), nuclear DAPI (Blue).

FIG. 10A-10G depicts 2D Differentiation of 4 different iPSC lines to iECs with human GMP sera AB and GMP cytokines (GMP medium). FIG. 10B-10C shows developing GMP-grade iEC differentiation medium in 4 different iPSC lines. Flow analysis for co-expression of endothelial markers CD31/CD144 was performed in 4 different cell lines. Our results show no significant difference between research-grade media and GMP-grade alternative.

FIG. 10D shows representative Immunocytochemistry images of endothelial markers CD31, VEGFR2, CD144, VWF, proliferation marker Ki67 and smooth muscle marker SM22 at day 21 post differentiation. iECs were differentiated on laminin 421 from vascular progenitors using basal GMPLV2 medium that was supplemented by either FCS+ Research-grade cytokines (GMPLV2) or Human GMP-grade AB serum+ GMP cytokines (GMP). CD31 (Red), VEGFR2, CD144, VWF, ki67 and SM22 (green), nuclear DAPI (Blue).

FIG. 11A-11B shows Validation of lower limit of pluripotent marker POU5F1 (OCT4) detection compared with housekeeping RPL13A gene using ddqPCR. cDNAs from iPSCs, day 4, day 11 and day 21 of differentiation were analyzed by ddqPCR to detect expression level of pluripotent marker OCT4. Day 11 and 21 iECs were induced either by GMPLV2 (supplemented with fetal Bovine serum plus research-grade cytokines) or GMPLV3 (Human AB serum plus research-grade cytokines). HUVECS (Human Umbilical Veins) were used as control endothelial cells. iPSCS and PBMCs (Peripheral Blood Mononuclear Cells) were used as positive and negative controls for OCT4 expression respectively. As shown in the graphs, OCT4 expression was significantly reduced in iECs from day 11 and 21 of differentiation, which indicates the removal of iPSCs during differentiation.

FIG. 12 (panels A-E) shows iPSC-derived vascular endothelial cell's (iECs) protein expression exhibit similar pattern of human umbilical vascular endothelial cells (HUVECs). A) PCA analysis show that iEC and HUVECs are cluster together and iPSC cluster is totally distinct cluster B) Heat map analysis also show the similarity between proteins expressed in iEC and HUVECs. C) Volcano plot shows top differentially expressed proteins HUVECs plus iEC versus iPSCs and HUVECs and iECs. D) Ven diagram of deferentially expressed proteins between iEC, iPSC and HUVECs. E) Enriched pathways in iECs and HUVECs.

FIG. 13 shows post translational modifications (PTMS) detected in iEC, iPSC and HUVECs.

FIG. 14 shows a schematic for differentiating iPSCs to iECs in 2D cultures with GMPLV1 medium supplemented with VEGF165, ANG1 & FSK. ICC results show endothelial differentiation with conditions supplemented with extra VEGF 165.

FIG. 15 shows validation of lower limit of pluripotent marker POU5f1 detection in research-grade cell lines. Also shown is detecting undifferentiated iPSCs from endothelial cells. Table of samples used for ddqPCR analysis and corresponding average POU5F1/RPL13A ratio in 4 different cell line. Graph of Average of 4 different cell lines POU5F1/RPL13A ratio in each critical step of iEC differentiation. Ratio is measured by dividing copy number of POU5F1 to copy number of housekeeping gene RPL13A. Day 21 iECs in all cell lines show near zero undifferentiated cells and probability of carcinogen formation.

FIG. 16 (panels A-I) shows iEC differentiation from GMP iPSC47b0-01 in small vessel. 2D differentiation of GMP iPSCs to iECs. A) Flow analysis of CD31 and CD144 positive cells on days 11 and 21 of differentiation. B) Representative Immunocytochemistry images of endothelial markers CD31, VEGFR2, CD144, VWF and functional assay Dil-AC-LDL uptake. C) Characterization of day 21 CD31 positive iECs with immunostaining using atrial markers Notch1, NRP1 and venous markers NRP2 and EphB4. D) Representative Immunocytochemistry images of day 21 iECs expressing Endothelial-Mesenchymal transition (EMT) markers Col1A and SM22. E) Immunocytochemistry images of iEC proliferation using proliferation marker Ki67 at day 21 post differentiation. CD31 (Red), all other antibodies (green), nuclear DAPI (Blue). F-I) Immunostaining analysis of markers mentioned in B-E.

FIGS. 17A-D show determination of initial iPS cell density for iEC differentiation for GMP iPSC 47b0-01 line. 17A) Schematic diagram for iEC differentiation. iPSCs were plated at 500, 1, 2 & 5K cells/cm2 on LN521. Cultures from all cell densities were dissociated and plated as 10 K/cm2 on laminin substrate LN421 on day 4. 17B) Phase contrast images of cultures from different cell density on days 4, 6, 11 and 14 of differentiation. 17C) Flow analysis of CD31 and CD144 positive cells from GMP iPSC 47b0-01 line with different iPSc seeding density on day 11 of differentiation. 17D) Table. Initial iPSC number affects fold expansion of differentiation products, Vascular progenitors and iECs. Vascular progenitors (day 4) and iECs (Day 11) fold expansion were calculated based on the ratio of harvesting to seeding cell densities in GMP-iPSC 47b0-01 cell line.

FIGS. 18A-18B show the detection of undifferentiated iPSCs from endothelial cells. 18A) Table of samples used for ddqPCR analysis and corresponding average LIN28 (pluripotency marker)/RPL13A ratio in GMP iPSC 47b0-01 RiEC in 2 separate preparations. 18B) Graph of Average of LIN28/RPL13A ratio from 2 different cell preparations of GMP iPSC 47b0-01 RiEC in each critical step of iEC differentiation. Ratio is measured by dividing copy number of LIN28 to copy number of housekeeping gene RPL13A. Day 21 iECs in all cell lines show near zero undifferentiated cells and probability of carcinogen formation.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

GMP iEC differentiation medium (e.g., used from about day 4 to about day 21) comprises the following:

| | |
|---|---|
| Supplement | Final concentration |

| Basal MV2 | | |
|---|---|---|
| PSA | 1:1000 | |
| AB serum (Human AB Serum, | 0.1 | ml/ml |
| Converted from Octaplas ®, | | |
| Pooled Plasma (Human) Xeno-Free, | | |
| Virus Inactivated) | | |
| EGF (Epidermal Growth Factor) | 5 | ng/ml |
| FGF2 (Basic Fibroblast Growth Factor) | 10 | ng/ml |
| Long R3 IGF-1 (Insulin- | 20 | ng/ml |
| like Growth Factor) | | |
| VEGF 165 (Vascular Endothelial | 260 | ng/ml |
| Growth Factor 165) | | |
| Ascorbic acid | 1 | ug/ml |
| Hydrocortisone | 0.2 | ug/ml |

TrypLE™ Express Enzyme refers to a dissociation enzyme—an animal origin free enzyme that cleaves peptide bonds on the C-terminal sides of lysine and arginine.

Accutase refers to a dissociation solution—a cell detachment solution of proteolytic and collagenolytic enzymes and does not contain mammalian or bacterial derived products.

An example of human recombinant laminin 421 is BioLamina LN421.

An example of human recombinant laminin 521 is BioLamina LN521.

An example of a cGMP feeder-free iPSC maintenance medium is mTeSR, which is a cGMP, feeder-free maintenance medium for human ES and iPS cells.

An example of a cGMP, enzyme-free human pluripotent stem cell selection and passaging reagent is ReLeSR™.

An example of an iPSC differentiation medium is STEMdiff™ APEL™2, which is a defined, animal component-free medium for differentiation of human ES and iPS cells to multiple lineage.

An example of Human AB serum is human serum lacking antibodies against A and B blood-type antigens that was converted from Pooled Plasma (Human), Solvent/Detergent Treated. An example of Pooled Plasma (Human), Solvent/Detergent Treated is Octaplas®.

Previously it was shown that hiPSC could differentiate to iECs in three steps of mesoderm, vascular progenitor and iEC inductions. Described herein we have further developed this protocol to be xeno-free and GMP-compatible.

We have demonstrated robust GMP iPSC differentiation to iECs in 5 different cell lines with high purity (94-99% double-expression of endothelial markers CD31/CD144 using flow analysis) without cell sorting. It is shown herein that iECs are functional by Dil-Ac-LDL uptake assay and endothelial specific Von Willebrand Factor (VWF) secretion. Less than 10% of cells were proliferative, majority of which co-expressed endothelial markers. Droplet digital PCR that detects low DNA concentration has shown pluripotency of near to zero in iECs. We have further applied an automated cell processing platform, CliniMACS Prodigy, to produce high cell number required for cell therapy.

A unique advantage of our invention is to produce functional GMP-grade iECs from iPSCs that could generate sufficient cells for clinical applications. Our GMP differentiation medium and extracellular matrix produces more than 95% of cells differentiated to iECs after 3 weeks of differentiation in 4 different cell lines.

Accordingly, various embodiments of the invention are based at least on these findings and advances.

Various embodiments of the invention provide for a method of generating endothelial cells from induced pluripotent stem cells (iECs), comprising
(i) differentiating induced pluripotent stem cells (iPSCs) into mesoderm cells by:
   removing cGMP feeder-free iPSC maintenance medium from plated induced pluripotent stem cells (iPSCs) and adding mesoderm induction medium, and
   culturing the iPSCs for about 1.5-2.5 days in about 36-38 C and in the presence of about 4-6% CO₂ to generate mesoderm cells;
(ii) differentiating the mesoderm cells into vascular progenitor cells by:
   removing the mesoderm induction medium and adding vascular progenitor medium, and
   culturing the cells for about 1.5-2.5 days in about 36-38 C and in the presence of about 4-6% CO₂ to generate vascular progenitor cells;
(iii) differentiating the vascular progenitor cells into iECs by:
   dissociating the vascular progenitor cells by using a first dissociation enzyme for about 4-6 minutes at 36-38 C and in the presence of about 4-6% CO₂, wherein the dissociation enzyme comprises an animal origin free enzyme that cleaves peptide bonds on the C-terminal sides of lysine and arginine;
   neutralizing the vascular progenitor cells by using basal endothelial cell growth medium MV2 plus about 9-11% human AB serum, spinning the vascular progenitor cell solution, removing the basal endothelial medium, and resuspending the vascular progenitor cells in GMP-grade iEC differentiation medium, wherein the human AB serum comprises human serum lacking antibodies against A and B blood-type antigens that was converted from Pooled Plasma (Human), Solvent/Detergent Treated;
   plating the vascular progenitor cells at about 9,000-11,000 cells/cm² with iEC differentiation medium onto a dish that was previously coated with 4-6 ug/ml human recombinant laminin 421 in PBS Mg+/Ca+ for about 1.5-2.5 hours at about 36-38 C and in the presence of about 4-6% CO₂,
   culturing the vascular progenitor cells at about 36-38 C and in the presence of about 4-6% CO₂ for about 6-8 days, feeding about every other day with iEC differentiation medium to generate iECs;
(iv) further culturing the iECs by:
   dissociating the iECs by using animal origin free enzyme that cleaves peptide bonds on the C-terminal sides of lysine and arginine (IX) for about 4-6 minutes at about 36-38 C and in the presence of about 4-6% CO₂;
   neutralizing the iECs using by using basal endothelial cell growth medium MV2 plus 9-11% Human AB serum, spinning the iEC solution, removing the basal endothelial medium, and resuspending the iECs in GMP-grade iEC differentiation medium, wherein the human AB serum comprises human serum lacking antibodies against A and B blood-type antigens that was converted from Pooled Plasma (Human), Solvent/Detergent Treated;
   plating the iECs at about 9,000-11,000 cells/cm² with iEC differentiation medium onto a dish that was previously coated with Sug/ml human recombinant laminin 421 in PBS Mg+/Ca+ for about 1.5-2.5 hours at about 36-38 C and in the presence of about 4-6% CO₂,
   culturing the iECs at about 36-38 C and in the presence of about 4-6% CO₂ for about 9-11 days, feeding about every other day with iEC medium, thereby generating iECs expressing CD31 and CD144.

In various embodiments, plating the vascular progenitor cells in steps (iii), (iv) or both, comprises plating the vascular progenitor cells at about 5,000-10,000 cells/cm², 10,000-15,000 cells/cm², 15,000-20,000 cells/cm², or 20,000-25,000 cells/cm², with iEC differentiation medium onto a dish that was previously coated with 4-6 ug/ml human recombinant laminin 421 in PBS Mg+/Ca+ for about 1.5-2.5 hours at about 36-38 C and in the presence of about 4-6% CO₂.

In various embodiments, the method further comprises dissociating the iECs that are generated in step (iv).

In various embodiments, the plated iPSCs were about 500-2500 iPSCs/cm². In various embodiments, the plated iPSCs were about 500-1000 iPSCs/cm². In various embodiments, the plated iPSCs were about 1000-2000 iPSCs/cm². In various embodiments, the plated iPSCs were about 1000 iPSCs/cm². In various embodiments, the plated iPSCs were about 1500 iPSCs/cm². In various embodiments, the plated iPSCs were about 2000 iPSCs/cm².

In various embodiments, the method further comprises providing plated iPSCs, wherein the plated iPSCs are made by:
dissociating iPSCs with a cGMP enzyme-free human pluripotent stem cell selection and passaging reagent for 5 minutes and neutralizing the iPSCs with iPSC maintenance medium;
triturating the iPSCs about 10-15 times to break large colonies of iPSCs to smaller colonies of iPSCs and to have a homogenous cell population that is about 60-200 um diameter per iPSC colony;
dissociating a subset of colony solution as single cells by using a dissociation solution, for 4-6 minutes at 36-38 C water bath, wherein the dissociation solution comprises a cell detachment solution of proteolytic and collagenolytic enzymes and does not contain mammalian or bacterial derived products;
neutralizing the dissociation solution by using a cGMP feeder-free iPSC maintenance medium, spinning the cell solution, removing the medium and resuspending the iPSCs in iPSC maintenance medium;
removing laminin solution from a plating dish that previously coated with 5 ug/ml human recombinant laminin 521 in PBS Mg+/Ca+ for 1.5-2.5 hours in 36-38 C and in the presence of 5% CO₂;
adding cGMP feeder-free iPSC maintenance medium to the plating dish;
adding about 500-2500 iPSCs/cm² to the plating dish and culturing at about 36-38 C and in the presence of about 4-6% CO₂;
changing the cGMP feeder-free iPSC maintenance medium about daily for an additional 1.5-2.5 days.

In various embodiments, about 1000-2000 iPSCs/cm² are added to the plating dish. In various embodiments, about 1000 iPSCs/cm² are added to the plating dish. In various embodiments, about 1500 iPSCs/cm² are added to the plating dish. In various embodiments, about 2000 iPSCs/cm² are added to the plating dish.

In various embodiments, the mesoderm induction medium comprises a final concentration of about 5-7 uM CHIR99021 and about 1:900-1:1100 Antibiotic-Antimycotic (PSA) in iPSC differentiation medium, wherein the iPSC differentiation medium comprises a defined, animal component-free medium for differentiation of human ES and iPS cells to multiple lineages.

In various embodiments, the vascular progenitor medium comprises final concentrations of about 20-30 ng/ml Bone Morphogenetic Protein 4 (BMP4), about 8-12 ng/ml FGF2, about 40-60 ng/ml VEGF165 and about 1:900-1:1100 Antibiotic-Antimycotic (PSA) in iPSC differentiation medium.

In various embodiments, the iEC differentiation medium comprises Basal MV2, PSA, Human AB Serum (AB serum), Epidermal Growth Factor (EGF), Basic Fibroblast Growth Factor (FGF2), Long R3 Insulin-like Growth Factor (IGF-1), Vascular Endothelial Growth Factor 165 (VEGF 165), Ascorbic acid, and Hydrocortisone.

In various embodiments, the iEC differentiation medium comprises Basal MV2, 1:900-1:1100 PSA, 0.05-0.15 ml/ml Human AB Serum (AB serum), 4-6 ng/ml Epidermal Growth Factor (EGF), 8-12 ng/ml Basic Fibroblast Growth Factor (FGF2), 18-22 ng/ml Long R3 Insulin-like Growth Factor (IGF-1), 240-280 ng/ml Vascular Endothelial Growth Factor 165 (VEGF 165), 0.5-1.5 ug/ml Ascorbic acid, and 0.1-0.3 ug/ml Hydrocortisone.

In various embodiments, the iEC differentiation medium comprises GMPLV1, and VEGF165, Forskolin (FSK), angiopoietin 1 (ANG-1), or a combination thereof.

In various embodiments, the iEC differentiation medium comprises GMPLV1, and VEGF165 (e.g., 200-230 ng/ml, 205 ng/ml, 210 ng/ml, 215 ng/ml), FSK (e.g., 20-30 uM, 22 uM, 25 uM, 28 uM), ANG-1 (e.g., 125-175 ng/ml, 140 ng/ml, 150 ng/ml, 160 ng/ml), or a combination thereof.

In various embodiments, the method comprises
(i) differentiating induced pluripotent stem cells (iPSCs) into mesoderm cells by:
   removing cGMP feeder-free iPSC maintenance medium from plated induced pluripotent stem cells (iPSCs) and adding mesoderm induction medium, and
   culturing the iPSCs for about 2 days in about 37 C and in the presence of about 5% CO₂ to generate mesoderm cells;
(ii) differentiating the mesoderm cells into vascular progenitor cells by:
   removing the mesoderm induction medium and adding vascular progenitor medium, and
   culturing the cells for about 2 days in about 37 C and in the presence of about 5% CO₂ to generate vascular progenitor cells;
(iii) differentiating the vascular progenitor cells into iECs by:
   dissociating the vascular progenitor cells by using a first dissociation enzyme for about 5 minutes at 37 C and in the presence of about 5% CO₂, wherein the dissociation enzyme comprises an animal origin free enzyme that cleaves peptide bonds on the C-terminal sides of lysine and arginine;
   neutralizing the vascular progenitor cells by using basal endothelial cell growth medium MV2 plus about 10% human AB serum, spinning the vascular progenitor cell solution, removing the basal endothelial medium, and resuspending the vascular progenitor cells in GMP-grade iEC differentiation medium, wherein the human AB serum comprises human serum lacking antibodies against A and B blood-type antigens that was converted from Pooled Plasma (Human), Solvent/Detergent Treated;
   plating the vascular progenitor cells at about 10,000 cells/cm² with iEC differentiation medium onto a dish that was previously coated with 5 ug/ml human recombinant laminin 421 in PBS Mg+/Ca+ for about 2 hours at about 37 C and in the presence of about 5% CO₂,
   culturing the vascular progenitor cells at about 37 C and in the presence of about 5% CO₂ for about 7 days, feeding about every other day with iEC differentiation medium to generate iECs;
(iv) further culturing the iECs by:
   dissociating the iECs by using animal origin free enzyme that cleaves peptide bonds on the C-terminal sides of lysine and arginine (1×) for about 5 minutes at about 37 C and in the presence of about 5% CO₂;
   neutralizing the iECs using by using basal endothelial cell growth medium MV2 plus 10% Human AB serum, spinning the iEC solution, removing the basal endothelial medium, and resuspending the iECs in GMP-grade iEC differentiation medium, wherein the human AB serum comprises human serum lacking antibodies against A and B blood-type antigens that was converted from Pooled Plasma (Human), Solvent/Detergent Treated;
   plating the iECs at about 10,000 cells/cm² with iEC differentiation medium onto a dish that was previously coated with 5 ug/ml human recombinant laminin 421 in PBS Mg+/Ca+ for about 2 hours at about 37 C and in the presence of about 5% CO₂,
   culturing the iECs at about 37 C and in the presence of about 5% CO₂ for about 10 days, feeding about every other day with iEC medium, thereby generating iECs expressing CD31 and CD144.

In various embodiments, the method further comprises dissociating the iECs that are generated in step (iv).

In various embodiments, the method further comprises providing plated iPSCs, wherein the plated iPSCs are made by:
dissociating iPSCs with a cGMP enzyme-free human pluripotent stem cell selection and passaging reagent for 5 minutes and neutralizing the iPSCs with iPSC maintenance medium;
triturating the iPSCs about 10-15 times to break large colonies of iPSCs to smaller colonies of iPSCs and to have a homogenous cell population that is about 60-200 um diameter per iPSC colony;
dissociating a subset of colony solution as single cells by using a dissociation solution, for about 5 minutes at about 37 C water bath, wherein the dissociation solution comprises a cell detachment solution of proteolytic and collagenolytic enzymes and does not contain mammalian or bacterial derived products;
neutralizing the dissociation solution by using a cGMP feeder-free iPSC maintenance medium, spinning the cell solution, removing the medium and resuspending the iPSCs in iPSC maintenance medium;
removing laminin solution from a plating dish that previously coated with about Sug/ml human recombinant laminin 521 in PBS Mg+/Ca+ for about 2 hours in about 37 C and in the presence of about 5% CO₂;
adding cGMP feeder-free iPSC maintenance medium to the plating dish;
adding about 2000 iPSCs/cm² to the plating dish and culturing at about 37 C and in the presence of about 5% CO₂;
changing the cGMP feeder-free iPSC maintenance medium about daily for about an additional 2 days.

In various embodiments, the mesoderm induction medium comprises a final concentration of about 6 uM CHIR99021 and about 1:1000 Antibiotic-Antimycotic (PSA) in iPSC differentiation medium, wherein the iPSC differentiation medium comprises a defined, animal component-free medium for differentiation of human ES and iPS cells to multiple lineages.

In various embodiments, the vascular progenitor medium comprises final concentrations of about 25 ng/ml Bone Morphogenetic Protein 4 (BMP4), about 10 ng/ml FGF2, about 50 ng/ml VEGF165 and about 1:1000 Antibiotic-Antimycotic (PSA) in iPSC differentiation medium.

In various embodiments, the iEC differentiation medium comprises Basal MV2, PSA, Human AB Serum (AB serum), Epidermal Growth Factor (EGF), Basic Fibroblast Growth Factor (FGF2), Long R3 Insulin-like Growth Factor (IGF-1), Vascular Endothelial Growth Factor 165 (VEGF 165), Ascorbic acid, and Hydrocortisone.

In various embodiments, the iEC differentiation medium comprises Basal MV2, 1:1000 PSA, 0.1 ml/ml Human AB Serum (AB serum), 5 ng/ml Epidermal Growth Factor (EGF), 10 ng/ml Basic Fibroblast Growth Factor (FGF2), 20 ng/ml Long R3 Insulin-like Growth Factor (IGF-1), 260 ng/ml Vascular Endothelial Growth Factor 165 (VEGF 165), 1 ug/ml Ascorbic acid, and 0.2 ug/ml Hydrocortisone.

In various embodiments, the iEC differentiation medium comprises GMPLV1, and VEGF165, Forskolin (FSK), angiopoietin 1 (ANG-1), or a combination thereof.

In various embodiments, the iEC differentiation medium comprises GMPLV1, and VEGF165 (e.g., 200-230 ng/ml, 205 ng/ml, 210 ng/ml, 215 ng/ml), FSK (e.g., 20-30 uM, 22 uM, 25 uM, 28 uM), ANG-1 (e.g., 125-175 ng/ml, 140 ng/ml, 150 ng/ml, 160 ng/ml), or a combination thereof

In various embodiments, the iEC differentiation method differentiated at least 90% of the cells into iECs; for example, after about 3 weeks of differentiation. In various embodiments, the iEC differentiation method differentiated at least 91%, 92%, 93%, 94%, or 95% of the cells into iECs; for example, after about 3 weeks of differentiation. In various embodiments, the iEC differentiation method differentiated more than 95% of the cells into iECs; for example, after about 3 weeks of differentiation.

In various embodiments, the iEC made by the method of the present invention have upregulation of EMT and glycolysis as compared to established human vascular EC line (HUVEC) cells.

In various embodiments, the iEC made by the method of the present invention have proteins relating to nucleic acid metabolism (e.g., SAMHD1 and DDX58), TNF-alpha signaling via NF-κβ (e.g., DDX58 and SERPINB2), and IL-6 and IL-10 signaling (e.g., HMOX1) upregulated as compared to established human vascular EC line (HUVEC) cells.

In various embodiments, the iEC made by the method of the present invention have a lower a dual phosphorylation (S56) and lysine monomethylation (K64) site on the protein AKT2 as compared to HUVECs vs iECs.

Various embodiments provide for iECs made by any one of the methods as described herein. In various embodiments, the iECs are provided in a clinically meaningful quantity. Clinically meaningful quantity is a quantity of iECs that for which is considered meaningful to patients, clinicians, or both. For example, a quantity of iECs that can be utilized to treat a patient in need thereof.

In various embodiments, the iEC differentiation medium comprises GMPLV1, and VEGF165, Forskolin (FSK), angiopoietin 1 (ANG-1), or a combination thereof.

In various embodiments, the iEC differentiation medium comprises GMPLV1, and VEGF165 (e.g., 200-230 ng/ml, 205 ng/ml, 210 ng/ml, 215 ng/ml), FSK (e.g., 20-30 uM, 22 uM, 25 uM, 28 uM), ANG-1 (e.g., 125-175 ng/ml, 140 ng/ml, 150 ng/ml, 160 ng/ml), or a combination thereof

In various embodiments, the iEC made by the method of the present invention have upregulation of EMT and glycolysis as compared to established human vascular EC line (HUVEC) cells.

In various embodiments, the iEC made by the method of the present invention have proteins relating to nucleic acid metabolism (e.g., SAMHD1 and DDX58), TNF-alpha signaling via NF-κβ (e.g., DDX58 and SERPINB2), and IL-6 and IL-10 signaling (e.g., HMOX1) upregulated as compared to established human vascular EC line (HUVEC) cells.

In various embodiments, the iEC made by the method of the present invention have a lower a dual phosphorylation (S56) and lysine monomethylation (K64) site on the protein AKT2 as compared to HUVECs vs iECs.

Various embodiments of the invention provide for a method of treating a condition in a subject in need thereof, comprising administering iECs made by made by a method of the present invention as described herein to the subject in need thereof.

In various embodiments, the condition comprises, transplantation, tissue reconstruction, ischemic trauma or injury, diabetes, cancer, or cosmetic condition.

In various embodiments, treating a condition comprises treating a wound, treating an amputation, treating a damaged organ, or treating necrosed tissue.

In various embodiments, the iEC made by the method of the present invention have upregulation of EMT and glycolysis as compared to established human vascular EC line (HUVEC) cells.

In various embodiments, the iEC made by the method of the present invention have proteins relating to nucleic acid metabolism (e.g., SAMHD1 and DDX58), TNF-alpha signaling via NF-κβ (e.g., DDX58 and SERPINB2), and IL-6 and IL-10 signaling (e.g., HMOX1) upregulated as compared to established human vascular EC line (HUVEC) cells.

In various embodiments, the iEC made by the method of the present invention have a lower a dual phosphorylation (S56) and lysine monomethylation (K64) site on the protein AKT2 as compared to HUVECs vs iECs.

Various embodiments of the invention provide for a method of drug screening, comprising: contacting a test drug to a population of iECs made by a method of the present invention as described herein; measuring one or more parameters of the iECs; and identifying the drug as a candidate based on the measured one or more parameters. Parameters of iECs include but are not limited gene expression, protein expression, and protein phosphorylation.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### cGMP Compliant iEC Differentiation

iPSC Preparation

### Day −3

### Coating:

Coat dishes with Sug/ml BioLamina LN521 in PBS Mg+/Ca+ for 2 hours in 37 C and 5% CO₂ incubator.
After 2 hr incubation, keep laminin solution in the dish at RT until plating cells
   Plating iPSCs:
Check iPSCs confluency. It should be between 70 to 80%
Dissociate iPSCs with ReLeSR for 5 minutes and neutralize it with iPSC maintenance medium mTeSR.
Triturate cells several times (10-15 times) to beak large colonies to smaller ones and have homogenous cell population (60-200 um diameter)
We plate cells as small colonies, but we need to count cell numbers for GMP purposes. To do so, we need to dissociate subset of colony solution as single cells by Accutase for 5 minutes at 37 C water bath.
Neutralize Accutase solution by mTeSR, spin cell solution, remove media and resuspend in mTeSR. Count cell number by Orflo.
Calculate the required volume of original cell solution to have 2 K/cm² iPSCs in the plating dish.
Remove laminin solution from plating dish and add mTeSR medium. Add cell solution to the dish and incubate it in in 37 C and 5% CO₂ incubator.
Change mTeSR medium daily for another 2 days.

### iEC Differentiation

### Day 0

Make Mesoderm induction medium by adding final concentration of 6 uM CHIR99021 and 1:1000 Antibiotic-Antimycotic (PSA) to STEMdiff™ APEL™2 Medium.
Remove mTeSR medium and replace it with Mesoderm induction medium.
Incubate cells with this medium for 2 days in 37 C and 5% CO₂ incubator.

### Day 2

Make Vascular progenitor medium by adding final concentrations of 25 ng/ml BMP4 (Bone Morphogenetic Protein 4), 10 ng/ml FGF2, 50 ng/ml VEGF165 and 1:1000 Antibiotic-Antimycotic (PSA) to STEMdiff™ APEL™2 Medium.
Remove Mesoderm induction medium and replace it with Vascular progenitor medium.
Incubate cells with this medium for 2 days in 37 C and 5% CO₂ incubator.

### Day 4

### Coating:

Coat dishes with Sug/ml BioLamina LN421 in PBS Mg+/Ca+ for 2 hours in 37 C and 5% CO₂ incubator.
After 2 hr incubation, keep laminin solution in the dish at RT until plating cells.

### Plating Vascular Progenitor Cells:

Dissociate Vascular progenitor cells by TrypLE™ Express Enzyme (1×) for 5 minutes in 37 C and 5% CO₂ incubator.
Neutralize cells by basal Endothelial cell growth medium MV2 plus 10% Human AB serum Converted from Octaplas® (AB Serum), spin cell solution, remove media and resuspend in GMP-grade iEC differentiation medium.
Count cell number by Orflo and calculate the volume of cell suspension required for 10 K/cm² cell in the dish.
Remove laminin solution and add iEC differentiation medium plus 10 k/cm² cell suspension.
Incubate cells in 37 C and 5% CO₂ incubator for 7 days and feed every other day with iEC differentiation medium.
For QC assay, plate 30,000 cells per well of 96 well plate on day 4 to be fixed and immunostained on day 11.

### Day 11

### Coating:

Coat dishes with Sug/ml BioLamina LN421 in PBS Mg+/Ca+ for 2 hours in 37 C and 5% CO₂ incubator.
After 2 hr incubation, keep laminin solution in the dish at RT until plating cells.
   Plating iECs:
Plating iECs on day 11 is following the same protocol as day 4
Incubate cells in 37 C and 5% CO₂ incubator for 10 days and feed every other day with iEC differentiation medium.
For QC assay:
   plate 30,000 cells per well of 96 well plate on day 11 to be fixed and immunostained on day 21.
   Perform LDL assay for checking iEC function.
   Fix and immunostain cells plated om day 4 with endothelial markers, smooth muscle markers and proliferation markers
   Run flow analysis to check percentage of cells that differentiated to iECs using endothelial markers CD31 and CD144.

### Day 21

Dissociation protocol is the same as day 11
For QC assay:

| GMP iEC differentiation medium | | |
|---|---|---|
| (from day 4 to day 21) | | |
| Supplement | Final concentration | |
| Basal MV2 | | |
| PSA | 1:1000 | |
| AB serum (Human AB Serum, | 0.1 | ml/ml |
| Converted from | | |
| Octaplas ®, Pooled Plasma | | |
| (Human) Xeno-Free, Virus | | |
| Inactivated) | | |
| EGF (Epidermal Growth Factor) | 5 | ng/ml |
| FGF2 (Basic Fibroblast | 10 | ng/ml |
| Growth Factor) | | |
| Long R3 IGF-1 (Insulin- | 20 | ng/ml |
| like Growth Factor) | | |
| VEGF 165 (Vascular Endothelial | 260 | ng/ml |
| Growth Factor 165) | | |
| Ascorbic acid | 1 | ug/ml |
| Hydrocortisone | 0.2 | ug/ml |

Fix and immunostain cells that plated on day 11 with endothelial markers, smooth muscle markers and proliferation markers
Perform LDL assay for checking iEC function.
Run flow analysis to check percentage of cells that differentiated to iECs using endothelial markers CD31 and CD144.

Proteomic analysis shows that iECs protein expression exhibit similar pattern of human umbilical vascular endothelial cells (HUVECs).

We noticed that initial iPSC colony size is important factor for successful iEC differentiation. Big colonies could not differentiate well. Center of such colonies remains undifferentiated due to the limited access to differentiation factors. These iPSC cells proliferate more than differentiated cells during differentiation period and reduce the percentage of endothelial cells in the culture.

We also checked the efficiency of differentiation using single cells instead of colonies. Single cells differentiation is much lower than colonies.

Using different cell density titrations (1, 2, 5 K iPSC/cm²) on Matrigel coating, we checked confluency and colony sizes in 7-day period after seeding by Sartorius Incucyte live cell imaging system.

We found that 1 K/cm² and 2 K/cm² iPSCs induce better differentiation as measured by Immunocytochemistry and flow analysis on day 11.

Next, we examined different GMP-compatible laminin substrates with different iPSC cell densities to see best differentiation efficiency. 1 K/cm² and 2 K/cm² iPSCs plated on LN521 and then upon dissociation of cells on days 4 and 11, plated on LN421 yields higher percentage of endothelial positive markers.

### Example 2

Differentiating iPSCs to iECs in 2D cultures with GMPLV1 medium supplemented with VEGF165, ANG1 & FSK.

| Condition | Concentrations |
|---|---|
| GMPLV1 | |
| GMPLV1 + VEGF (GMPLV2) | 210 ng/ml VEGF |
| GMPLV1 + ANG-1 | 150 ng/ml ANG-1 |
| GMPLV1 + VEGF + | 210 ng/ml VEGF, |
| ANG-1 | 150 ng/ml ANG-1 |
| GMPLV1 + FSK | 25 uM FSK |
| GMPLV1 + VEGF + FSK | 210 ng/ml VEGF, 25 uM FSK |
| GMPLV1 + FSK + ANG-1 | 25 uM FSK, 150 ng/ml ANG-1 |
| GMPLV1 + VEGF + | 210 ng/ml VEGF, 150 ng/ml |
| ANG-1 + FSK | ANG-1, 25 uM FSK |

### Example 3

### Proteomics of iPSC-Derived Vascular Endothelial Cells Reveal Extensive Similarity with an Immortalized Human Endothelial Cell Line

### Methods

iPSC generation. The iPSC lines utilized in this study were generated from healthy lean (BMI<27 kg/m²) male and female controls by the iPSC Core at Cedars-Sinai Biomanufacturing Center. These control iPSC lines were generated from the peripheral blood mononuclear cells (PMBCs) utilizing non-integrating oriP/EBNA1-based episomal plasmid vectors, as described in (Rajamani et al, 2018). This approach resulted in <5% of abnormal karyotypes of iPSCs. All undifferentiated iPSCs were maintained in mTeSR⁺ medium (StemCell Technologies) onto BD Matrigel™ matrix-coated plates. The cell lines used in this study are summarized at Table 1. The reprogramming of iPSCs and differentiation protocols were carried out in accordance with the guidelines approved by Stem Cell Research Oversight committee (SCRO) and IRB, under the auspices of IRB-SCRO Protocols Pro00036896 (Sareen Stem Cell Program) and Pro00032834 (iPSC Core Repository and Stem Cell Program).

TABLE 1
Characterization of the iPSC lines utilized in this study

| | Parent | Disease | | | |
|---|---|---|---|---|---|
| Subject | cell line | condition | Sex | Age | Race |
| CS0007iCTR-n07 | PBMC | Control | Male | 60 | Unknown |
| (abbreviated | | | | | |
| as 07n07) | | | | | |
| CS0002iCTR-n11 | PBMC | Control | Male | 51 | Unknown |
| (abbreviated | | | | | |
| as 02n11) | | | | | |
| EDi028-A | PBMC | Control | Male | 78 | White |
| CS00031CTR-n14 | PBMC | Control | Male | 34 | Asian |
| (abbreviated | | | | | |
| as 03n14) | | | | | |
| EDi042-A | PBMC | Control | Female | 79 | White |

* PBMC: Peripheral Blood Mononuclear Cell

Differentiation of vascular endothelial cells from iPSCs (iECs). For the generation of vascular endothelial cells (ECs) from iPSCs (iECs), we have adapted a previous protocol (Harding et al, 2017) to make our “in-house” robust and efficient protocol. Briefly, iPSCs (OCT4 expression>90%) were plated in planar onto Matrigel-coated plates as small colonies of cells, and 3 days later they were induced to mesoderm (ME—Phase I) using CHIR99021(6 μM, Xcess Bio) for 2 days. Next, vascular progenitors (VP—Phase II) were generated using a combination of BMP4 (25 ng/ml, R&D Systems), FGF2 (10 ng/ml, PeproTech) and VEGF165 (50 ng/ml, PeproTech) for another 2 days. After that, when the majority of the VPs presented a cobblestone-like morphology in the periphery of the original iPSC colonies, cells were dissociated with Accutase (Sigma), and the cells in the periphery that easily lifted were re-plated in planar onto Matrigel-coated plates at a density of 10,000 cells/cm² with VEGF₁₆₅ (50 ng/ml) and Y-27632 (10 μM, StemCell Tech) to induce EC progenitors (ECP-Phase III) for 7 days, changing media every other day. For purification and maturation of iECs (Phase IV), iECs were dissociated at Day 11 and re-plated at the same cell density onto Matrigel-coated plates with VEGF₁₆₅(50 ng/ml) and media was changed every other day for 10 days. This process was repeated on Day 21 and extended for another 10 days if necessary. The base media used for Phases I and II was STEMdiff™ APEL™2 medium (StemCell Tech) and for Phases III and IV, EC Growth medium MV2 (ECGM-MV2) (PromoCell). For imaging purposes, cells were re-plated onto Matrigel-coated 96-well plates at a density of 100,000 cells/cm² at Day 4 for imaging on Day 11 or plated at Day 11 for imaging on Day 21. Human umbilical vein endothelial cells (HUVECs) were used as a positive control in some experiments and were fed with iEC Phase IV complete media as described above.

Tube formation in vitro assay. For the tube formation assay, iECs Day 21 were dissociated and re-plated onto a solid layer of Matrigel in 96-well plates (15,000 cells/well) using Phase IV complete media plus Y-27632. BF images were taken 24, 48, and 72 hours after.

Dil-Acetylated LDL-uptake assay. Dil fluorescent dye-labeled acetylated low-density lipoprotein (Dil-ac-LDL) (10 g/ml, Cell Applications) was added to Phase IV medium of iECs at Day 11 or Day 21 and incubated for 4 hours at 37° C., as described previously (Harding et al, 2017). Cells were then washed with PBS, fixed, and stained with DAPI. Images were taken with ImageXpress Micro XLS (Molecular Devices) and analyzed using ImageJ. HUVECs fed with Phase IV medium were used as positive control, and iPSCs fed with mTeSR⁺ medium were used as negative control.

Immunofluorescence. Cells were first fixed with 4% paraformaldehyde (PFA) in phosphate-buffered saline (PBS) for 20 minutes and subsequently washed 2× with PBS. Fixed cells were then permeabilized and blocked for 1 hour in a “blocking buffer” containing PBS with 10% donkey serum (Millipore) and 0.1% Triton-X (Bio-Rad). Primary antibodies were diluted in the blocking buffer and kept on cells overnight at 4° C. The following primary antibodies and dilutions were used: VEGFA (1:100, Abcam), CD31 (1:100, Cell Signaling), CD144 (1:100, Abcam), VGFR2 (1:100, Cell Signaling). The next day, after thorough washing using PBS with 0.1% Tween-20 (ThermoFisher), cells were incubated with appropriate species-specific Alexa Fluor-conjugated secondary antibodies (ThermoFisher) diluted in the blocking buffer (1:1,000) for 1 hour at room temperature. After washing in PBS with 0.1% Tween-20, cells were incubated in DAPI diluted in PBS (1:2,500) for 15 min. Immunofluorescence images were taken using appropriate fluorescent filters using ImageXpress Micro XLS and analyzed using ImageJ Software.

Real Time qPCR analysis. Relative gene expression was quantified using Real time-qPCR. For this, cells were washed with PBS and the total RNA was extracted and isolated using Quick-RNA MiniPrep kit (Zymo Research), according to the manufacturer's instructions. The concentration and purity of RNA were determined by spectrophotometric analysis (NanoDrop, ThermoFisher), and all samples had a A_{260/280} ratio around 2.0 (Desjardins and Conklin, 2010). Afterwards, RNA (1 μg) was treated with DNAse (ThermoFisher), and then reverse transcribed to cDNA with oligo(dT) using the High-Capacity cDNA Reverse Transcription kit (ThermoFisher). Real-time qPCR was performed in three replicates using SYBR Green Mastermix (Applied Biosystems) and primer sequences to specific to each gene and run on a CFX384 Real Time system (Bio-Rad). Human RPL13 was used as a reference gene and relative expression was determined using 2^{-ΔΔ} CT method.

Flow cytometry analysis. Cells were singularized using Accutase and filtered using a 70 m nylon mesh. Cells were washed once with PBS plus 10% FBS (washing buffer), spun down at 300×g for 5 min (4° C.) and resuspended in the washing buffer for 20 min (blocking phase) on ice. After this, the following treatments were added for 30 min on ice: no antibodies for “Unstained samples”; FITC mouse anti-human CD31 and Alexa-Fluor® 647 mouse anti-human CD144 for “Double Stained samples”; FITC mouse IgG1 κ Isotype control and Alexa Fluor® 647 mouse IgG1 κ Isotype control for “Double ISO samples”. After this, cells were washed 3× in the washing buffer (300×g for 5 min each wash, 4° C.). Cells were then fixed with FBS+4% PFA for 15 min on ice. To finalize, cells were again washed 3× in the washing buffer and analyzed using FACS Attune NxT (ThermoFisher). Double ISO samples were used for gating.

MACS sorting analysis. Cells were singularized with Accutase, filtered using a 70 μm nylon mesh, and sorted on a MACS Sorting machine (Miltenyi Biotec) according to the manufacturer instructions. Briefly, cells were centrifuged at 300×g for 3 min and then resuspended with 60 μl iEC Phase IV complete media (as described above) plus 20 μl CD31 MicroBeads per 5×10⁶ cells, for 15 min on ice. After this, 1 ml of medium was added and cells were centrifuged again. Cells were resuspended in 1 ml of MACS sorting buffer and proceeded to magnetic separation using “Possel” for positive selection, following instructions of the manufacturer (AutoMACSPro, Miltenyi Biotec). After this, “CD31 positive fraction,” “CD31 negative fraction,” and “Unsorted cells” proceeded with CD144 staining flow cytometry analysis as described earlier.

Proteomic Sample Preparation and Mass Spectrometry Acquisition. Cell pellets were lysed using 8 M UREA/5% SDS with 100 mM DTT. Protein concentration was determined by BCA assay and 50 ug of protein per sample was aliquoted for further processing. Protein aliquots were processed, digested, and cleaned using the S-TRAP system (Protifi). Peptides were dried following elution from the S-TRAP columns, and dried peptides were resuspended at a concentration of 1 μg/μL for injection onto MS. Four micrograms of protein per sample, suspended in 20 μL total loading volume, were injected onto a 15 cm Phenomenex Omega Polar C18 3 μm 100A 150×0.3 mm column and separated on a 4-45% gradient of Acetonitrile in 0.1% FA water at 7 μl/min flow rate over 60 minutes total separation time. Eluting peptides were ionized at the source of a Thermo Fisher Orbitrap Lumos mass spectrometer operating in Data Independent Acquisition mode. The instrument cycled continuously between 1) an intact MS1 scan of all peptides between 400-1600 m/z in the orbitrap detector at resolution 120K, accumulation time of 50 ms and target AGC of 400K and 2) 40 subsequent MS2 scans systematically isolating all ions within 15 mz range intervals from 400-1000 m/z and analyzing high energy induced collision (CE 30%) induced fragments between 200-2000 m/z from each window in the orbitrap at 30K resolution, maximum injection time of 54 per scan and target AGC set to 500K. Total cycle time to progress through each MS1 and 40 MS2 scan series was 3 seconds.

Proteomic analysis. Raw MS files were analyzed using the DIA-Neural Network platform (PMC 6949130), with files searched using a ‘library-free’ strategy via two approaches: Total protein analysis was done by searching files against an in silico digested protein FASTA sequence database (Uniprot Reviewed and Canonical human sequences); for PTM analysis, a library of detectable peptides was generated using the FragPipe workflow wherein the same raw files used for total protein analysis were used to generate pseudospectra that are then searched for presence of pre-specified PTMs. In this study, we interrogated phosphorylation of Threonine, Serine, and Tyrosine (T,S,Y), acetylation of lysines (K) and N-termini, and mono- and dimethylation of arginines (R) and lysines. High confident (FDR<1%) Pseudospectra identifications were consolidated into a library that was then used to perform a second round of DIA-Umpire quantitative extraction, and the quantified peak areas for each protein with identification qvalue <1% FDR were analyzed for differential abundance between cell types using Fragger (PMC5409104). For PTM analyses, an additional filter was applied to include only peptides with >0.8 MS1 correlation score, meaning that the MS1 chromatogram correlated in co-elution and shape with the MS2 fragments, thus minimizing chances that a called PTM peptide could be in reality the product of the unmodified form erroneously identified by any overlapping fragment patterns (not all of which may carry the mass shift for a given PTM). PTM validation was completed using Percolator. PTM abundances from the filtered DIANN peptide tables were normalized to the total protein quantification for the gene product that a given peptide was assigned to. Quantitative comparison of PTMs was done using Philosopher, IonQuant, and simple t tests in Excel (Microsoft). Total proteome analyses were performed in R. Principal Component Analysis was performed using the Prcomp package using default settings. Differential Expression analysis was performed using the DESeq2 bioconductor package, which was used to generate the data for volcano plots which were plotted using Ggplot2. Differential expression analysis was conducted on proteins with complete observations across all samples in the comparison (N=4028 proteins between iEC and HUVECs versus iPSC; N=4632 proteins between iEC and HUVEC comparison)

The Venn diagram was prepared by compiling a list of all proteins detected in more than 6 of the 9 iEC samples, 2 of the 3 HUVEC samples, and 5 of the 7 iPSC samples and then running set comparisons to determine which proteins were shared or absent across the three groups. A heatmap was prepared by plotting the expression of all proteins differentially expressed between iEC and HUVEC samples versus iPSC samples with an adjusted p-value of 0.05.

Statistical analyses. Data are presented as the mean±standard error of the mean (SEM). Statistical significance between groups was determined by One-way ANOVA followed by Dunnet post-test. Two-tailed paired Student's test was used as appropriate. P values <0.05 were considered statistically significant. Statistical analyses and graphs were generated using GraphPad Prism 7 for Windows Software (GraphPad Software).

### Results

Robust and efficient differentiation of iPSC-derived vascular endothelial cells across five different iPSC lines. Human iPSCs were induced to differentiate into non-tissue-specific pan-vascular endothelial cells (iECs). For this, pluripotent iPSCs were passaged as colonies using ReLeSR™ (StemCell Tech) and after 3 days, when they were small colonies of approximately 60-200 μm size cleaned out for any spontaneous differentiation, the differentiation cocktail was initiated to induce iPSCs into primitive mesoderm (Phase I) through the activation of the Wnt signaling pathway using CHIR99021, highly selective inhibitor of glycogen synthase kinase 3 (GSK-3), for 2 days. Afterwards, cells were induced into vascular progenitors (Phase II) using the same media, supplemented with additional morphogens and growth factors including BMP4, FGF2 and VEGF165 for another 2 days. At Day 4 of differentiation, two different cell populations were observed: vascular progenitors at the periphery of the colonies and less differentiated pluripotent cells in the center of the colonies. Cells were manually lifted (mostly the vascular progenitors at the periphery), singularized, and re-plated at a specified cell density (10,000-20,000 cells/cm²) with specific media to mature vascular progenitors into endothelial progenitors with supplementation of VEGF₁₆₅ for 7 days. At Day 11, endothelial progenitors were again dissociated and re-plated at similar cell densities with the same media for another 10 days to enhance maturation and purification into iECs. The elongated shape of iECs seen at Day 21 or later of differentiation is a characteristic of strongly expressing CD144-positive ECs as published previously (Dyer & Patterson 2010).

This simple multi-stage differentiation protocol was repeated across 5 different iPSC lines to show robustness and reproducibility of the protocol. EC markers CD31⁺/CD144⁺ at Day 21 were found across all 5 cell lines, and a high percentage of cells expressed both markers (>80% of double expression in all cell lines). Additional EC markers were probed, and all lines presented VEGFA⁺/CD31⁺ and VEGFR2⁺/CD31⁺ protein expression. Importantly, we tested EC functionality in vitro at Day 21 through the functional assay Acil-Dil-LDL uptake. LDL-uptake by the iECs at this stage was routinely >40%. To further probe functionality, iECs were dissociated and re-plated onto a thick layer of Matrigel using low cell density, and after 24 hours, iECs formed tube-like structures, which were maintained after 48 and 72 hours. These results show that we could establish an efficient and reproducible protocol to differentiate iPSCs into functional vascular iECs with high expression of the canonical vascular endothelial protein markers.

To understand the development of the iECs throughout the differentiation process, cells from multiple cell lines were collected in different days (iPSC stage (Day 0), Day 11 and Day 21), and gene expression of main vascular EC markers were probed. iECs from all cell lines presented a higher expression of PECAM-1 (CD31) and VE-CADHERIN (CD144) at Day 21 compared to Day 11, and most of the cell lines presented higher expression of KDR (VEGFR2) at Day 21 compared to Day 11. Importantly, iECs at Days 11 or 21 presented minimal or non-detectable mRNA expression of pluripotent marker OCT4 compared to iPSCs. These results show that the dissociating and extending the protocol from Day 11 to Day 21 further matured iECs. Gene expression of arterial (NRP1 and NOTCH1) and venous genes (EPHB4 and NRP2) assessed in EC cultures. By days 11 and 21 both sets of genes were upregulated in the iEC cultures, suggesting neither a distinct arterial nor venous phenotype of the iECs in vitro at these time points.

For a cGMP-compliant cell therapy to translate into the clinic, it is important that the final cell product be a defined homogeneous cell population. While iECs generated here have >80% CD144⁺/CD31⁺ expressing cells by Day 21, to reach the greater purity with homogenous population of iECs expressing endothelial cell markers, we established the feasibility of Magnetic Associated Cell Sorting (MACS) to enrich the final iECs population. Day 21 iECs MACS-sorted for CD31 were probed for CD144 expression by flow cytometry and re-plated for immunostaining analysis at Day 23 (CD144/CD31 expression). Unsorted iECs presented around 92% of CD144⁺ expression at Day 21. Upon CD31-based MACS, this purity increased to 99.7% CD144+iECs, showing the feasibility of cell sorting to generate pure population of iECs for cell therapy applications Sorted and unsorted iECs carried until Day 23 on differentiation continued to present high levels of CD144⁺ and CD31⁺ cells.

Proteomic analysis reveals iPSC-derived vascular ECs exhibit a similar protein profile as an established human vascular EC line (HUVEC cells). To gain knowledge from the iECs generated by this method and extensively define their protein expression profile, proteomic analysis was conducted on Day 21 iECs generated from 3 different iPSC lines (03n14, EDi028-A and EDi042-A). iPSCs (at Day 0) from the same lines were used as negative control, and HUVECs as a standard comparator. Prior to proteomics studies, we confirmed that HUVECs maintained an EC phenotype HUVECs presented high protein expression of main EC markers, such as VEGFA⁺/CD31⁺, VEGFR2⁺/CD31⁻ and CD144⁺/CD31⁺, and they were also functional as probed by LDL-uptake assay and Matrigel-based tube formation assay. Quantification of CD144⁺/CD31⁺ expression was also checked by flow cytometry and HUVECs presented around 99% of double expression. CD144 (VE-CADHERIN), CD31 (PECAM-1) and VEGFR2 (KDR) expression were also probed at the mRNA level by RT-qPCR, and HUVECs presented upregulation of both VE-CADHERIN and PECAM-1 compared to iPSCs. These results made us confident to use this cell line as a valid primary endothelial cell comparator in the proteomics analysis.

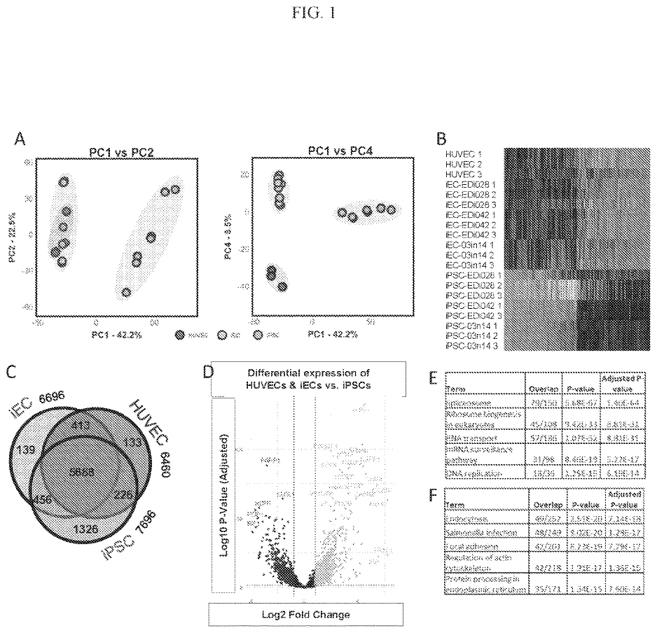

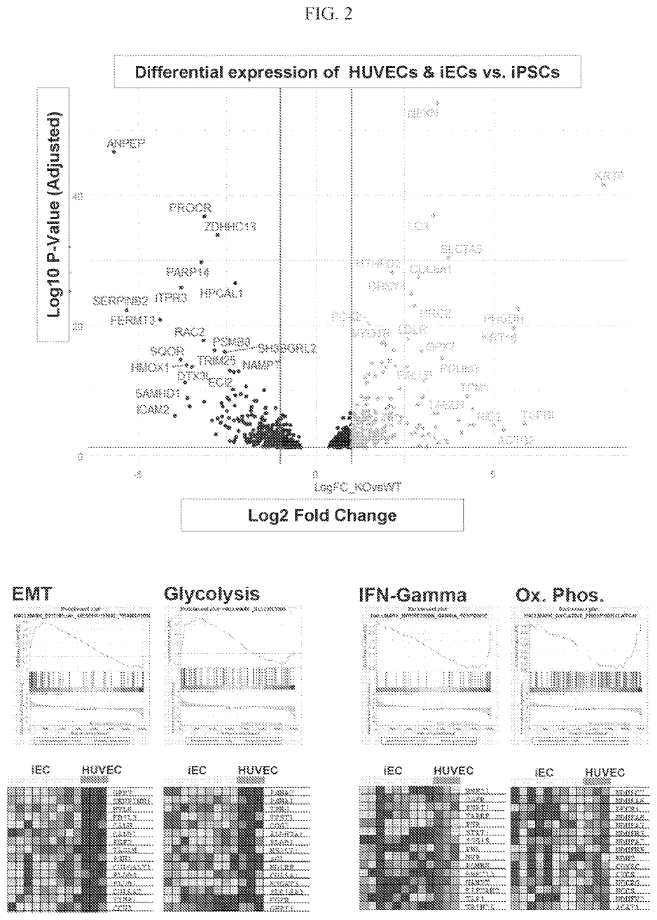

Principal Component Analysis (PCA) comparing iPSCs, iECs and HUVECs demonstrates a distinct separation of 2 groups when running PC1 vs PC2: Importantly, iECs are grouped with HUVECs and they both were far from iPSCs (FIG. 1A), demonstrating that iECs presented a similar protein expression pattern to HUVECs, and both of which are unique from the protein expression pattern of iPSCs. Not until PC5 did we achieve a distinct separation between iEC and HUVECs based on their proteomic profiles, implying that although they are endothelial cells, iPSC-derived ECs still present some unique protein expression that is distinct from HUVECs (FIG. 1A). The separation of protein expression pattern of iPSCs compared to iECs and HUVECs can also be observed on the heatmap generated at FIG. 1B, which supports the similarities between the protein expression pattern of HUVECs and iECs from 3 different cell lines. Interestingly, while a large proportion (N=5688) of proteins were detected within all the 3 cell types (FIG. 1C), 64.6% of the proteins showed statistically significant differential expressed proteins (DEPs). Interestingly, iECs have roughly equivalent numbers of shared proteins with HUVECs (413) or with iPSCs (456), and HUVECs have a much smaller number of proteins uniquely shared with iPSCs (226). iPSCs contain a largely unique proteome (1326). If we compare the 2 endothelial cell types (HUVECs and iECs), there are fewer proteomic differences between these cell types (13%) than between endothelial cell types and iPSCs (16% of proteins). These data indicate that iECs are more proteomically similar to HUVECs than to iPSCs, demonstrating the success of the differentiation process.

Differential expression of proteins unique to endothelial cell type and iPSCs can be examined using the volcano plot in FIG. 1D, including the upregulation of proteins related to cellular bioenergetics (APOE and PARP1) in endothelial cell types. The majority of the top 10 proteins upregulated in endothelial cell types are mini-chromosome maintenance (MCM) proteins involved in cell replication. In iPSCs, proteins related to cell survival (HSPB1) and intra-cellular regulations such as plasma membrane organization (EHD2) and signal transduction (MVP) were upregulated. To gain insight into functional implications of proteomic differences, we conducted a gene set enrichment analysis on the proteins upregulated in the iPSCs (FIG. 1E) as well as proteins upregulated in the endothelial cell types (FIG. 1F). The iPSC proteome was enriched with proteins involved in spliceosome and RNA transport whereas endothelial cell types expressed proteins enriched for focal adhesion and actin cytoskeleton regulation. Overall, comparison of proteomic differences between iPSC and EC types demonstrated expected results, highlighting proteins and pathways consistent with a pluripotent state in iPSCs.

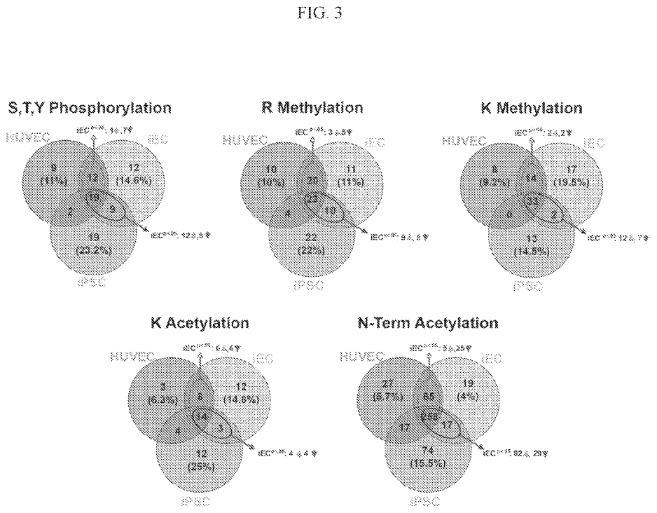

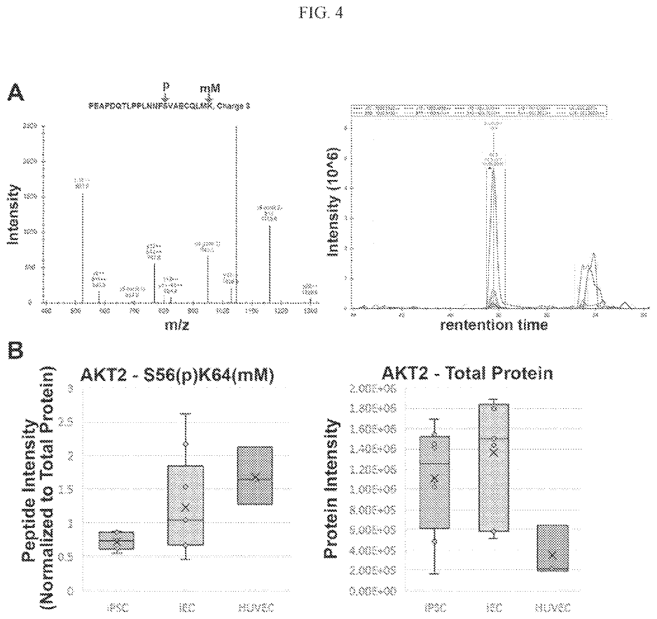

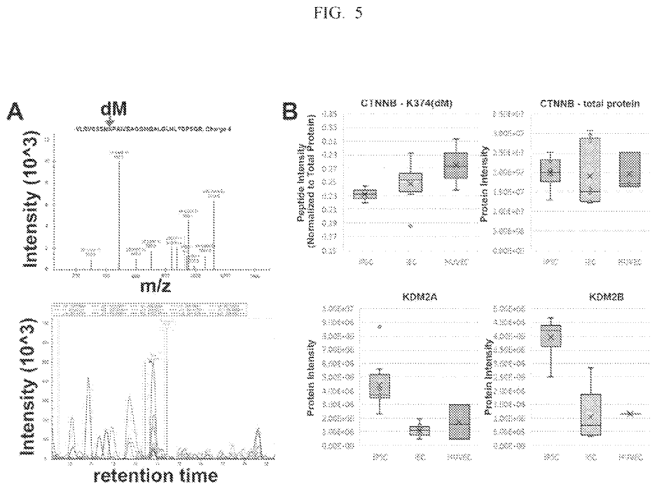

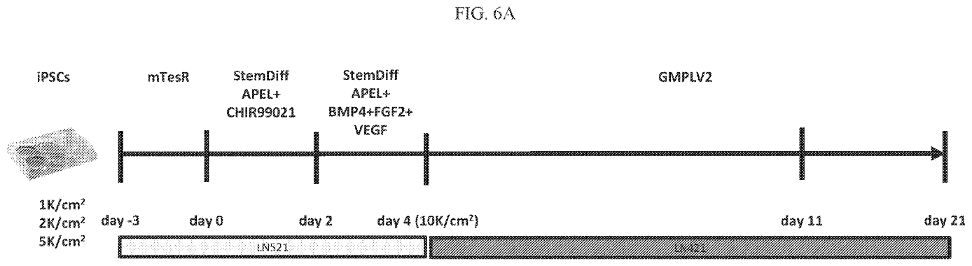

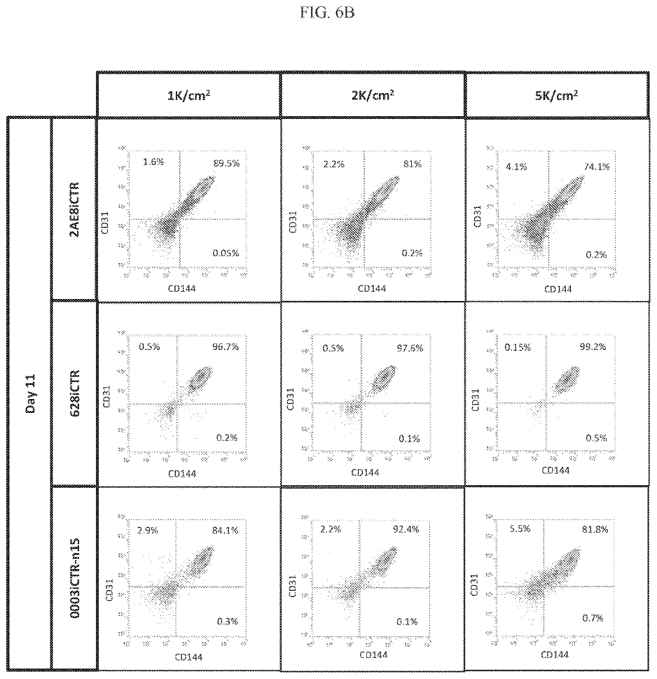

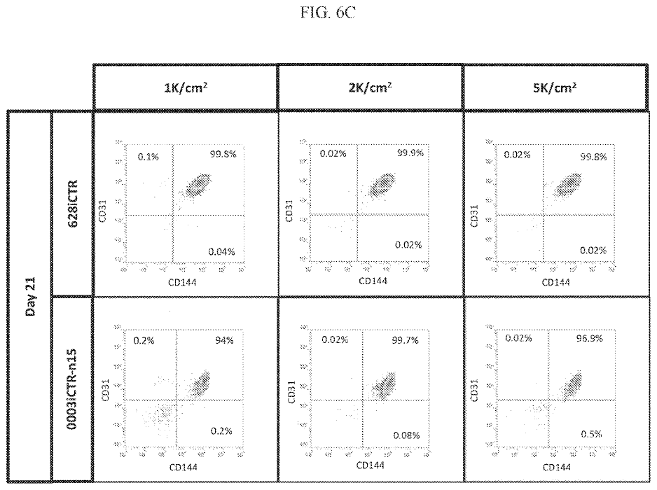

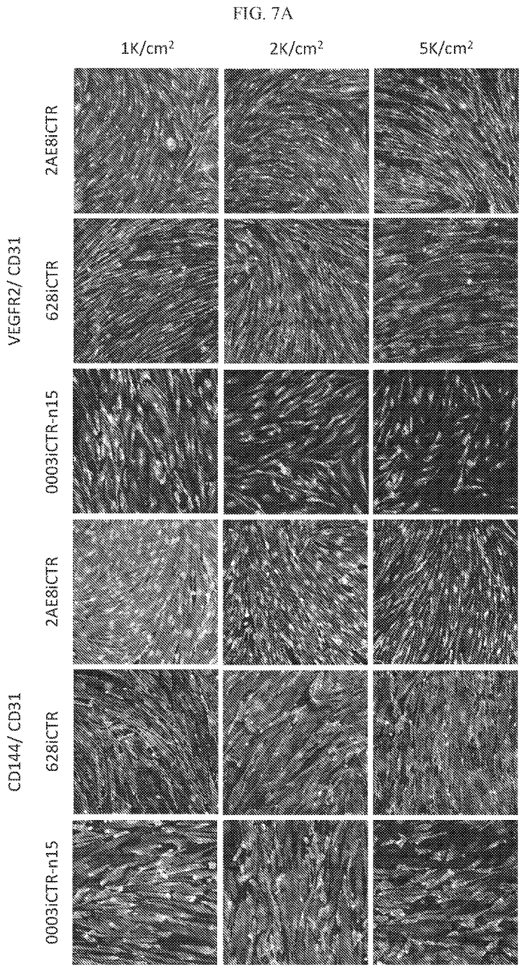

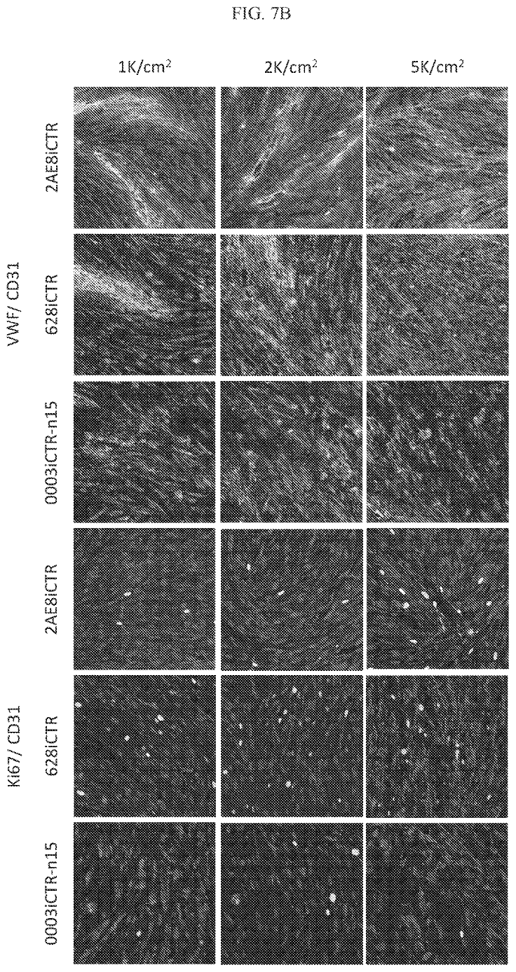

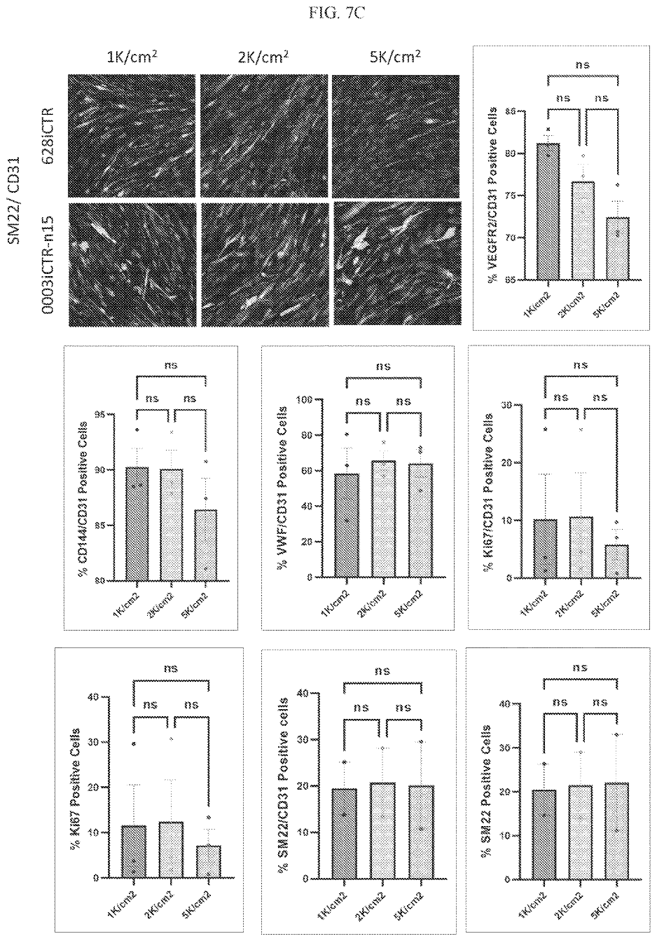

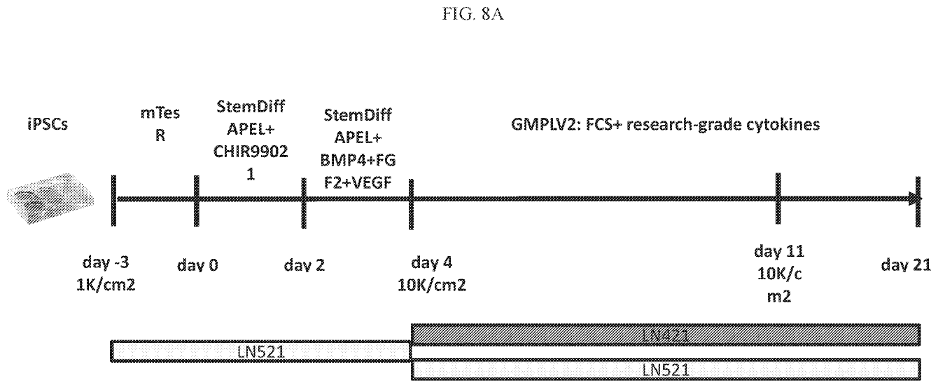

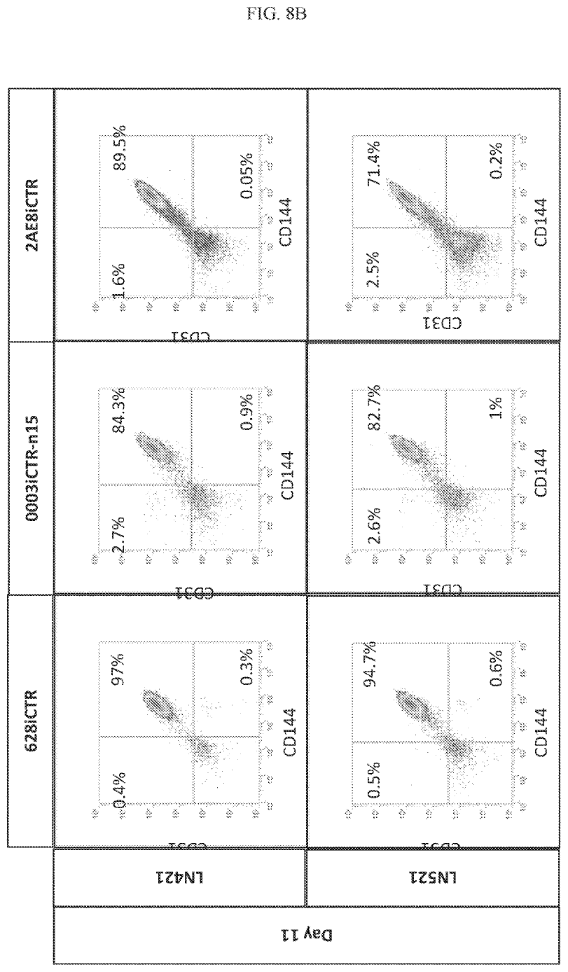

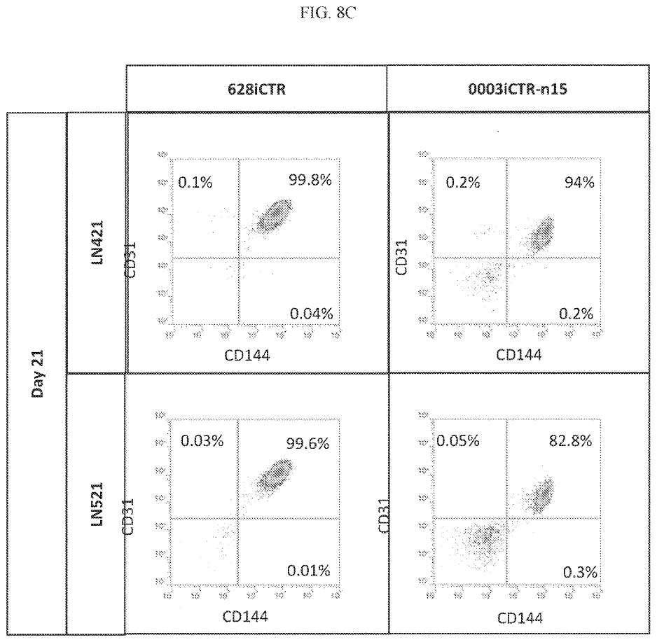

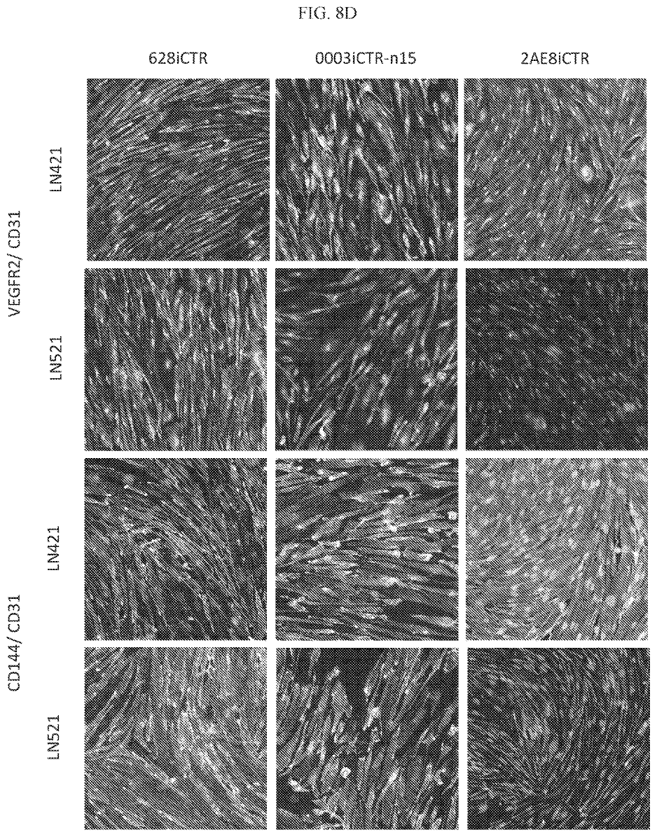

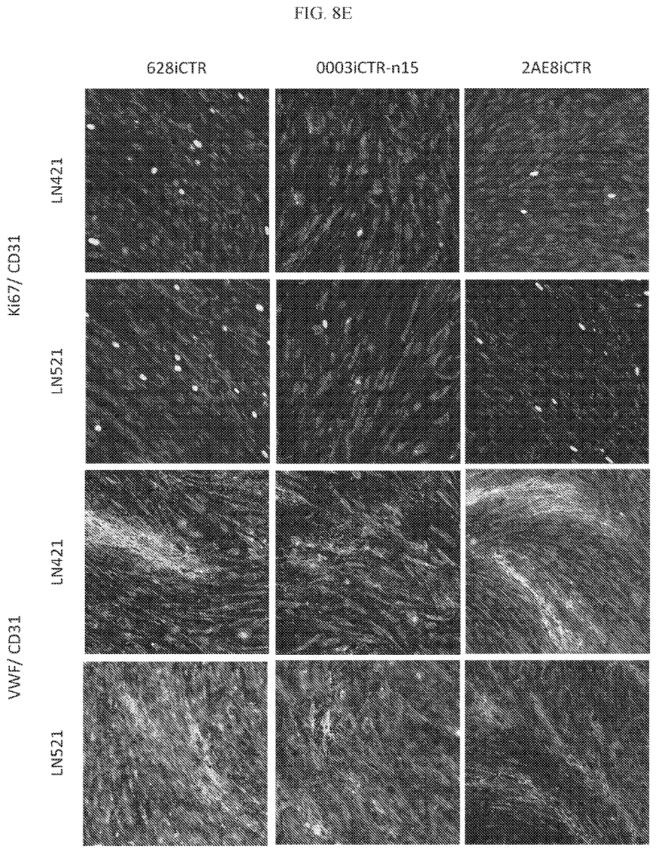

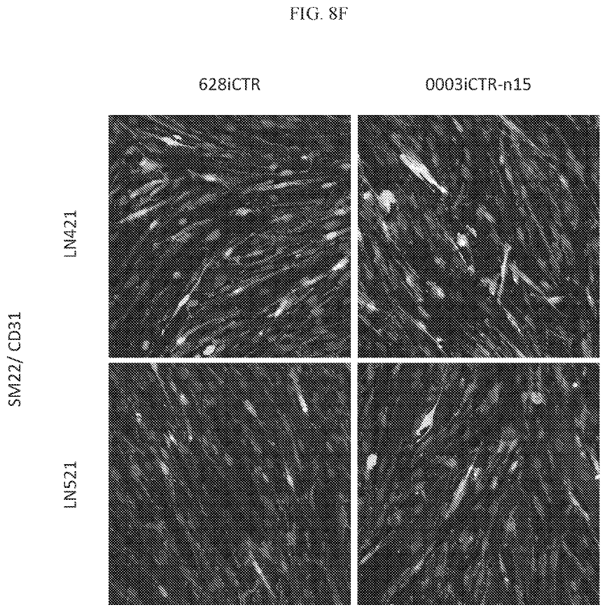

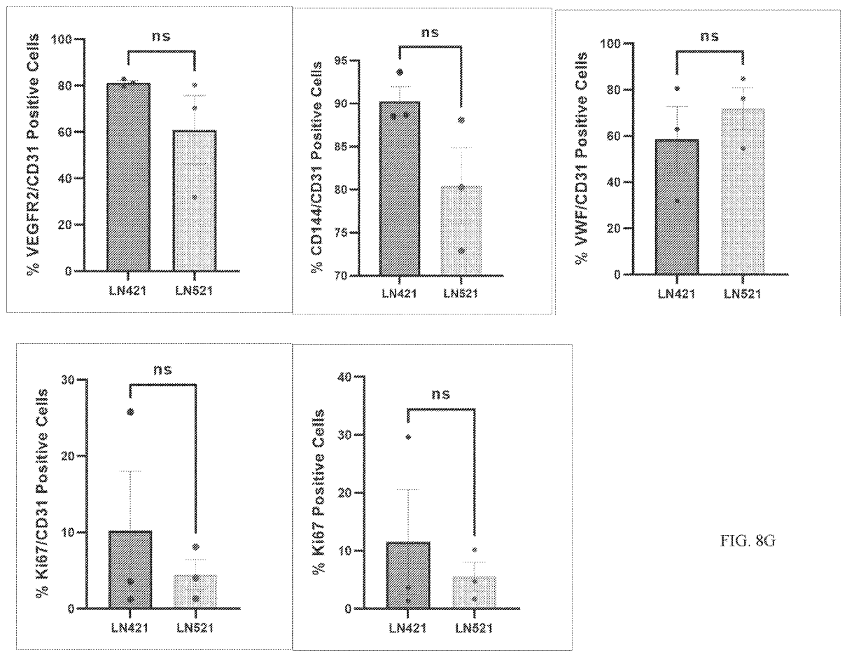

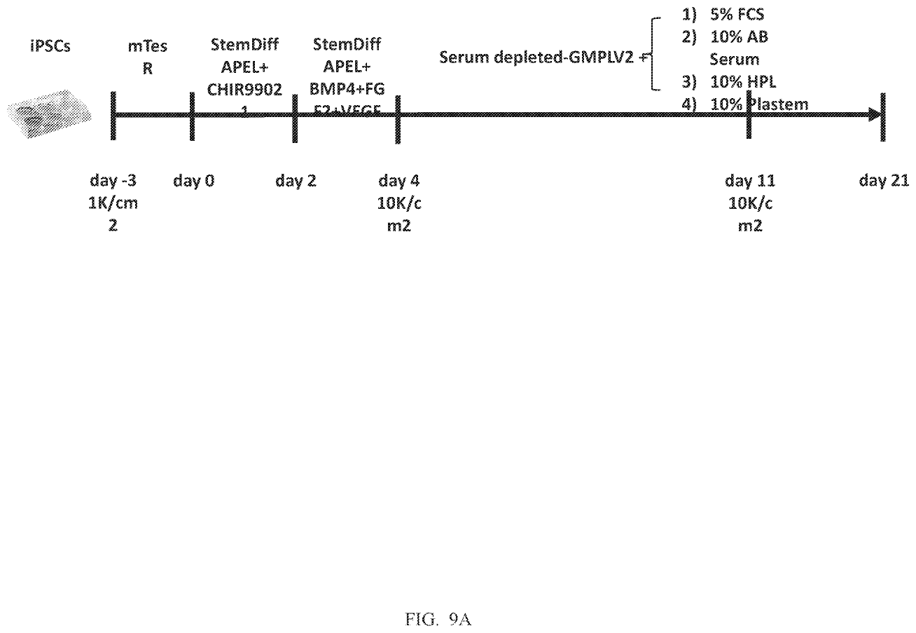

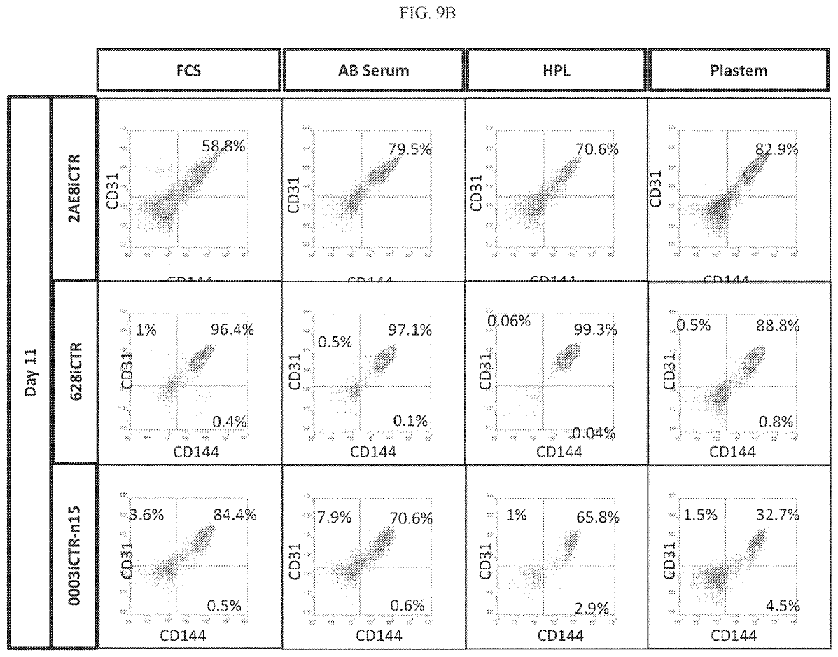

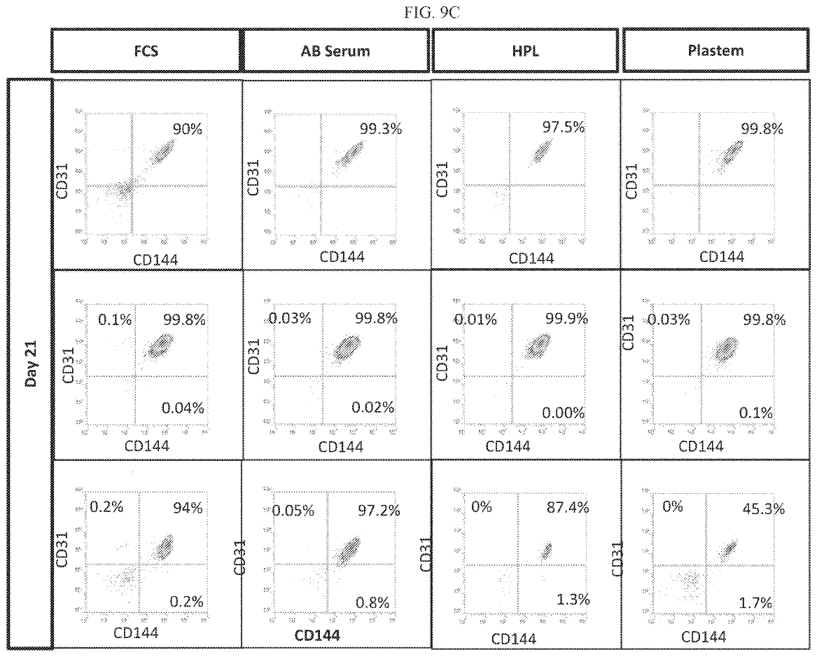

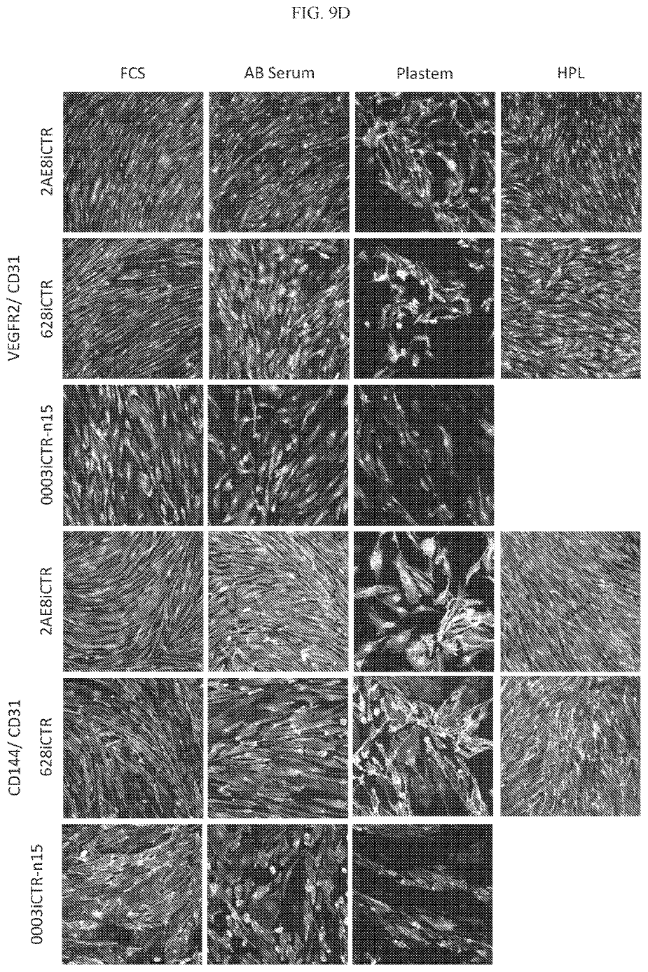

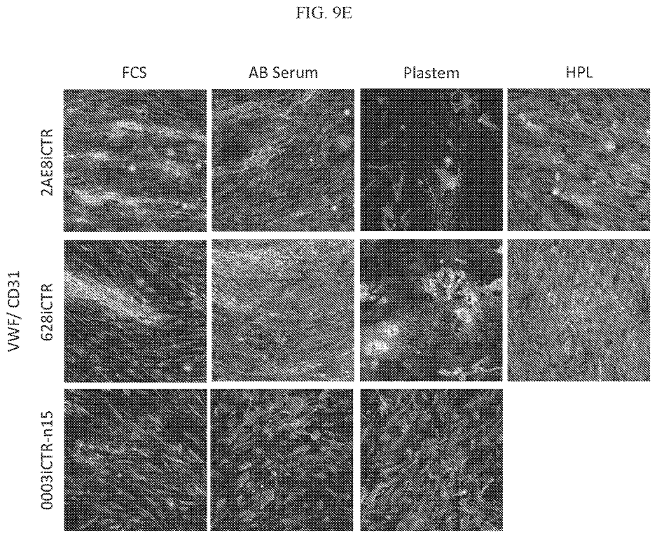

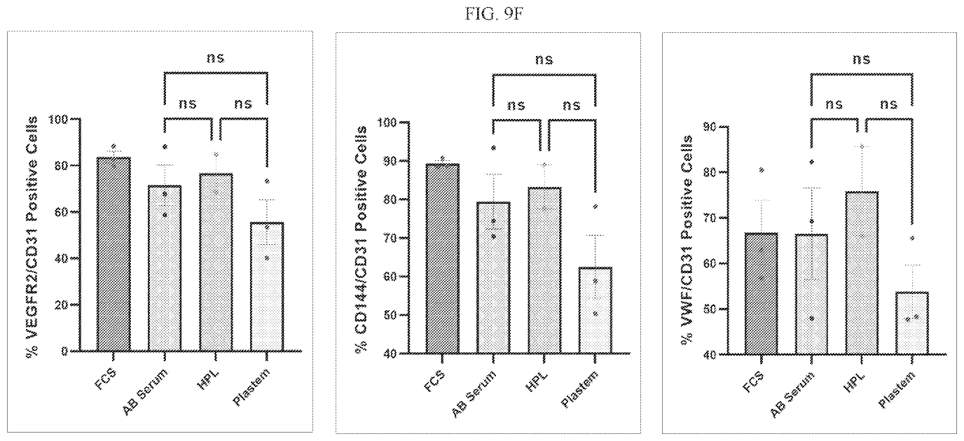

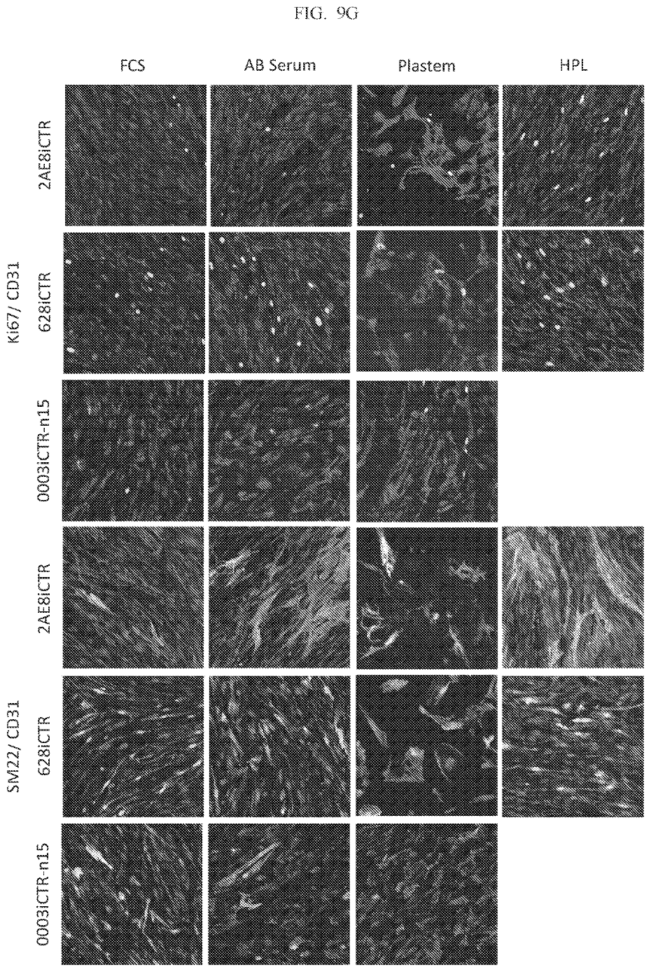

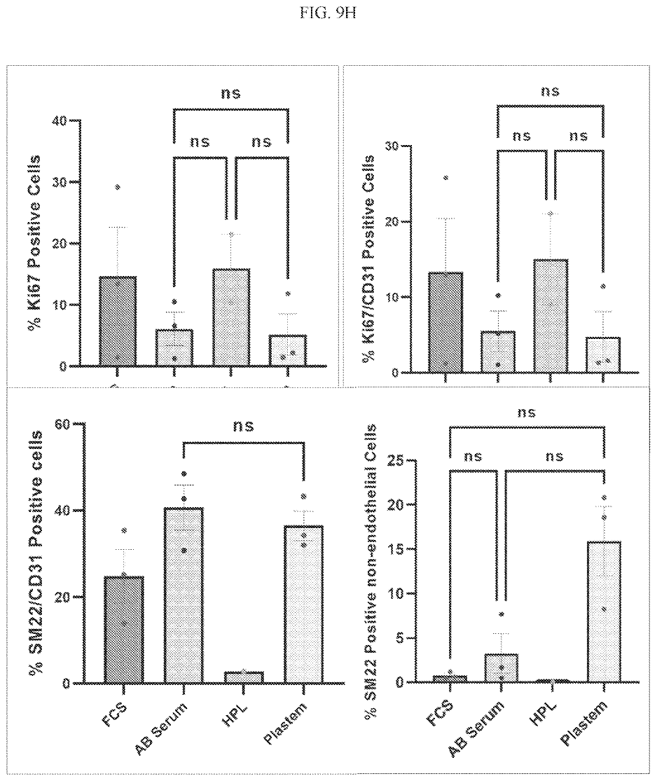

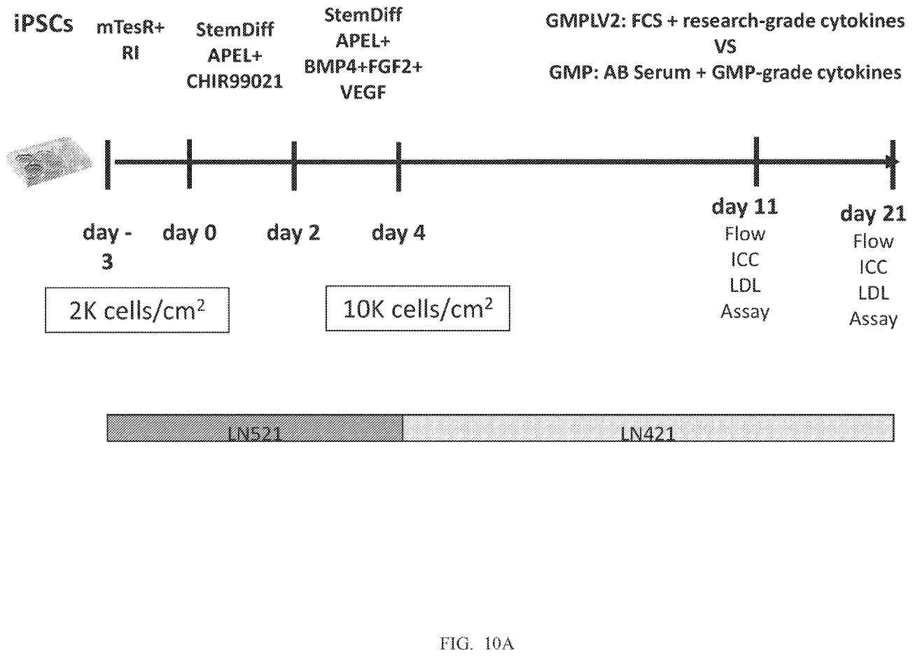

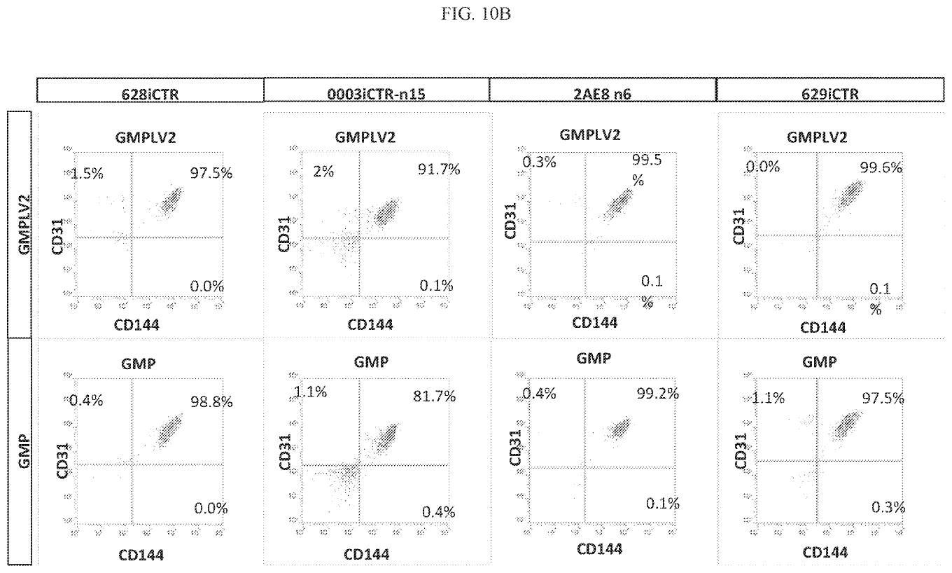

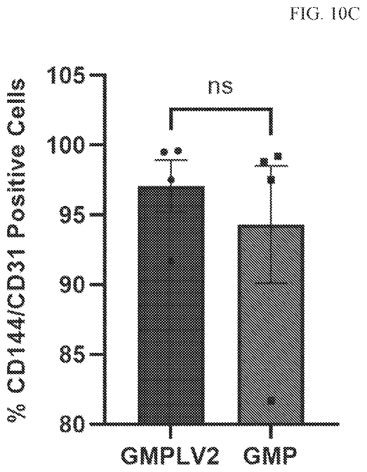

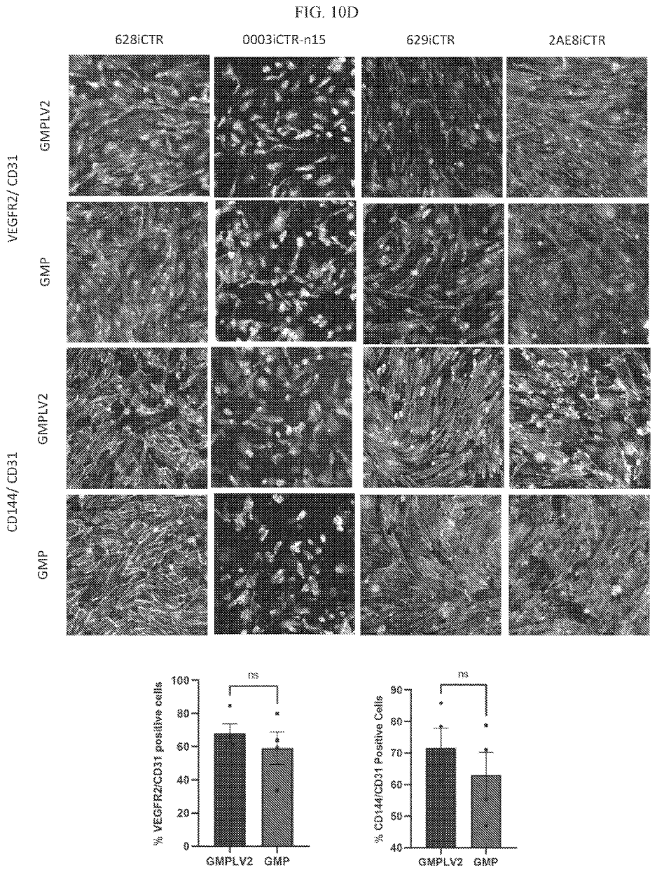

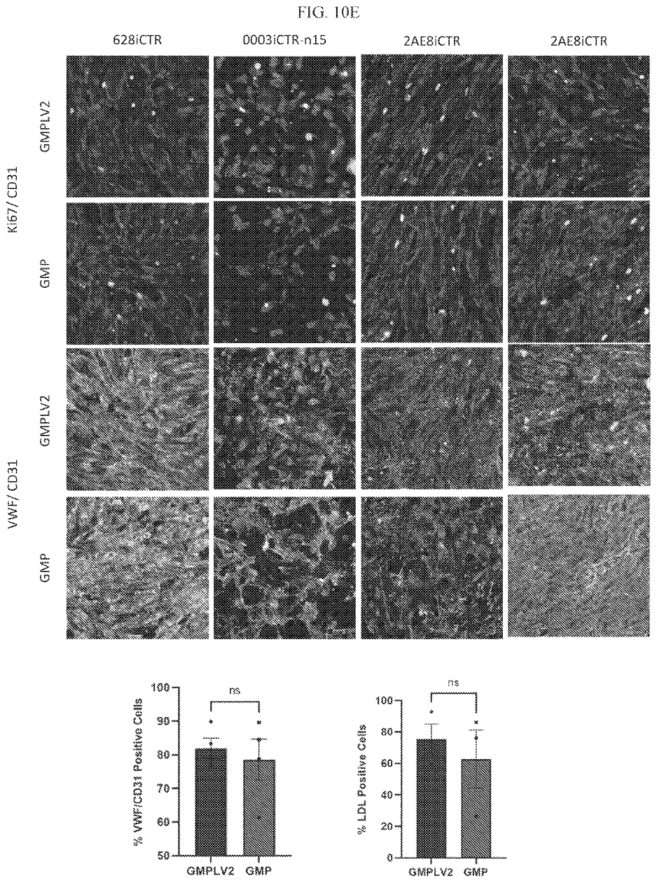

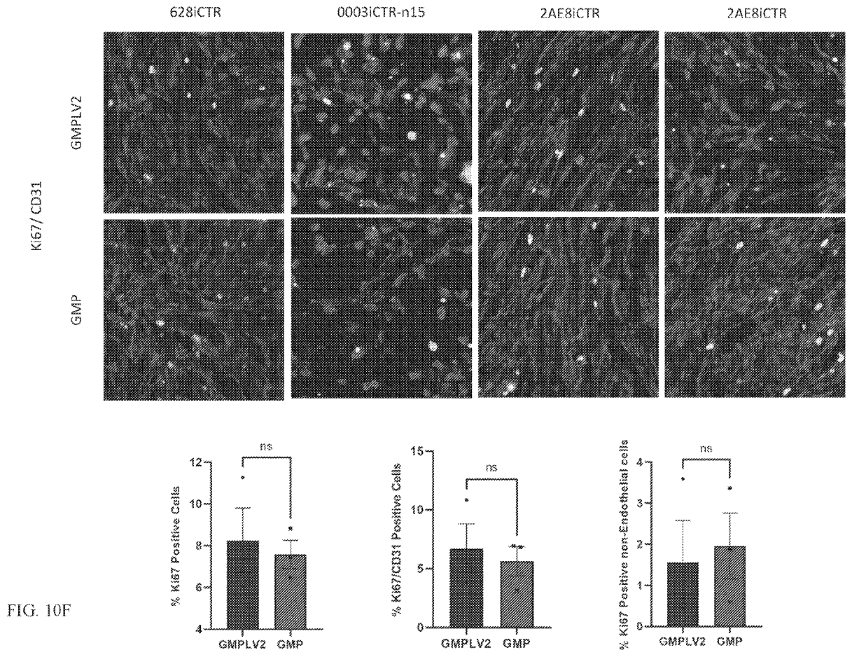

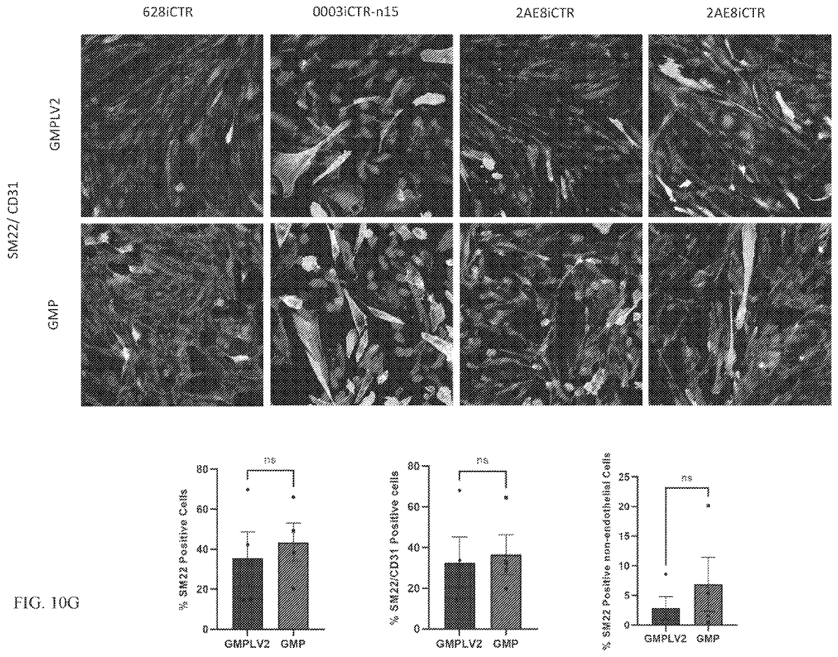

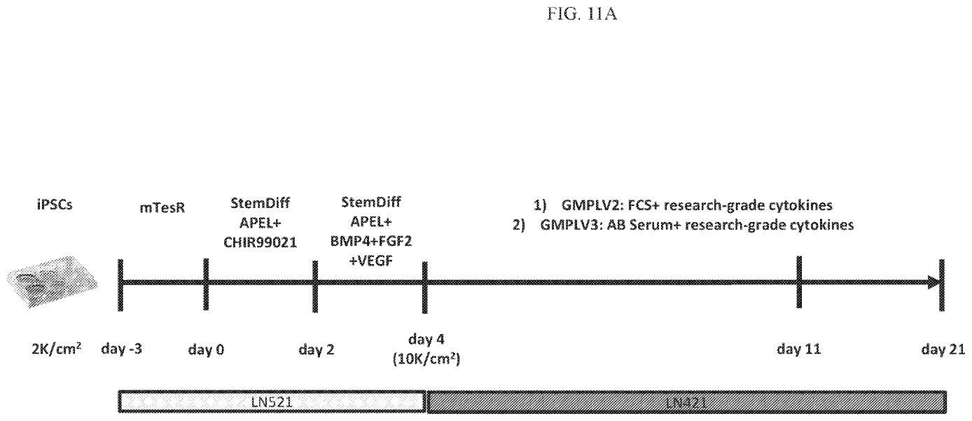

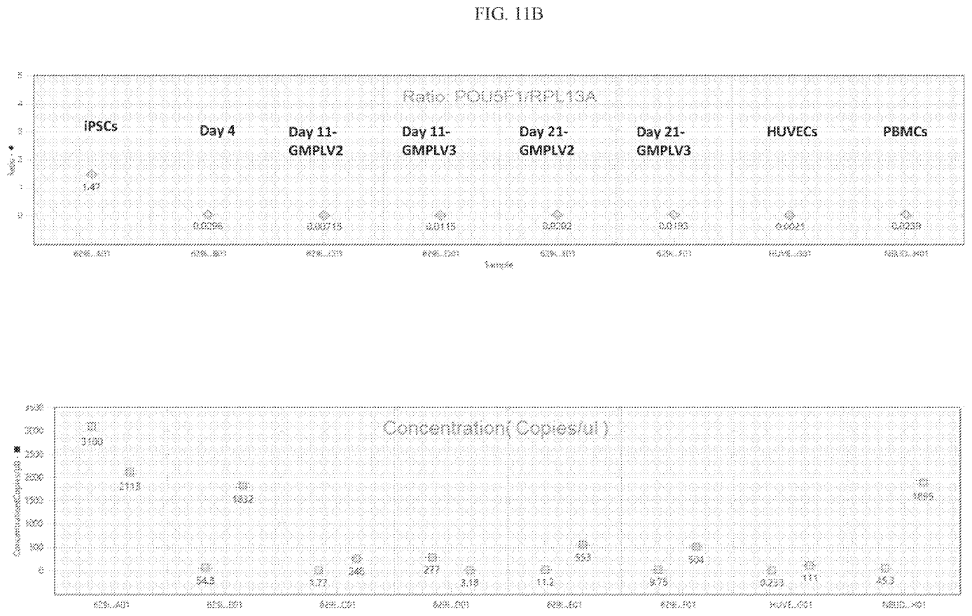

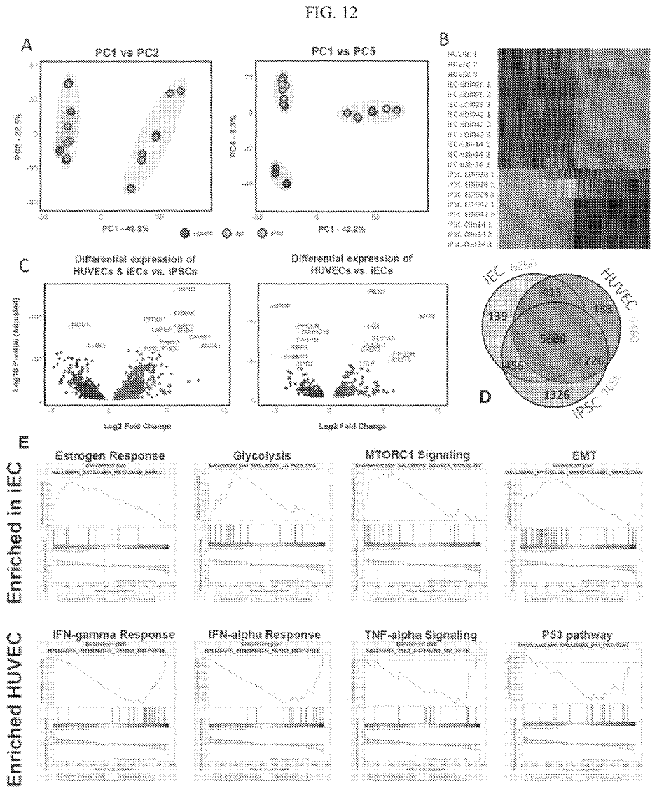

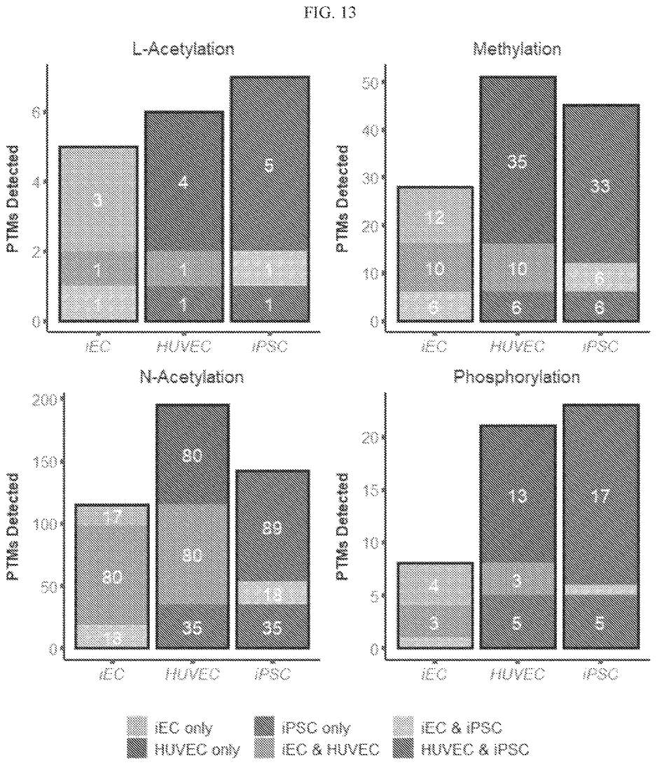

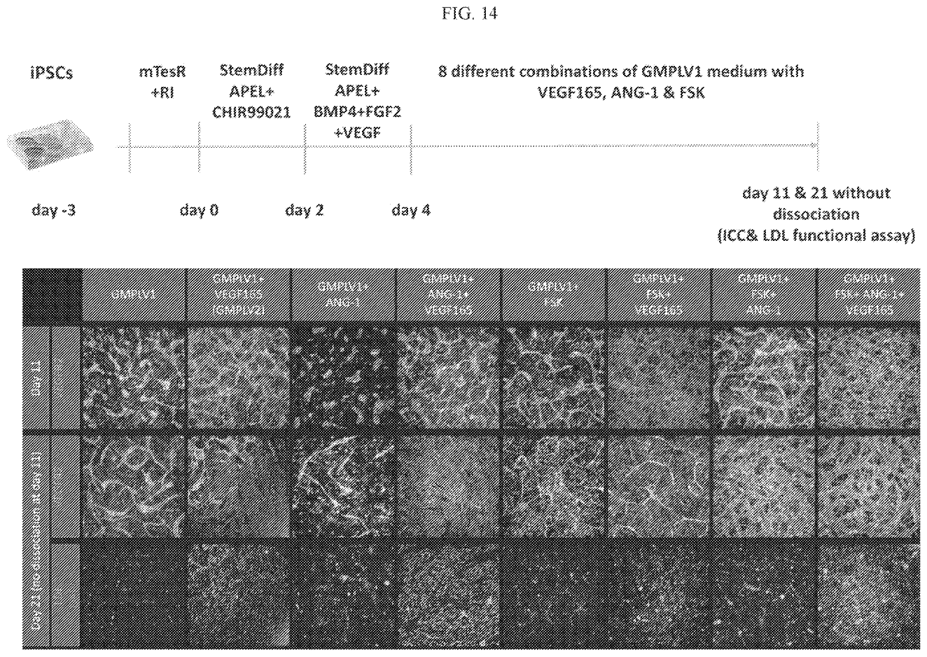

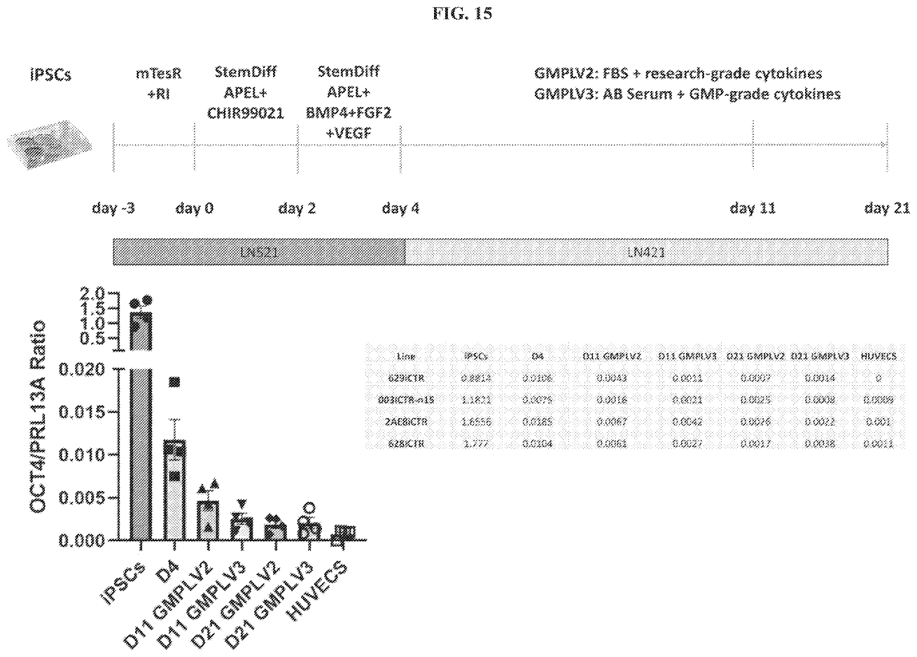

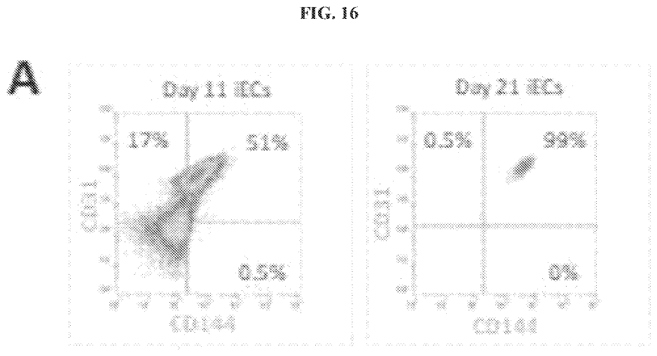

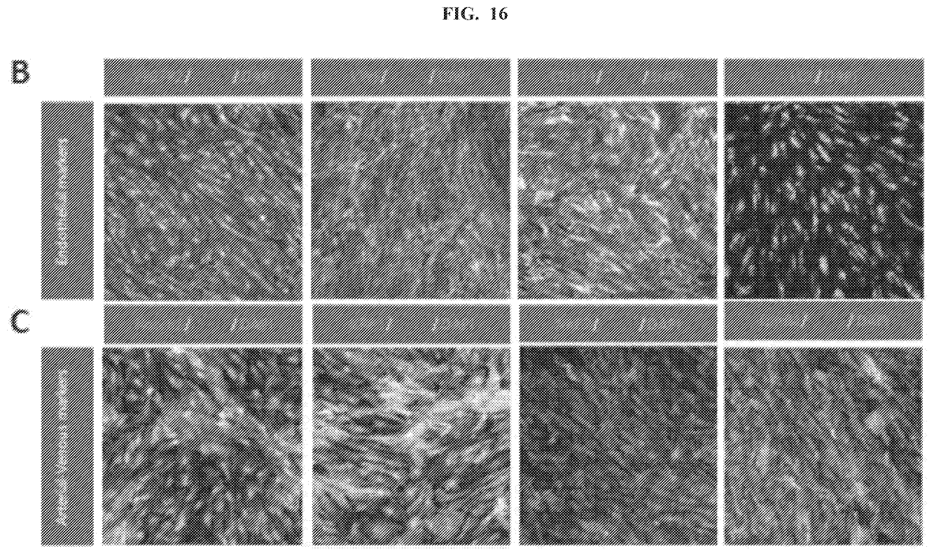

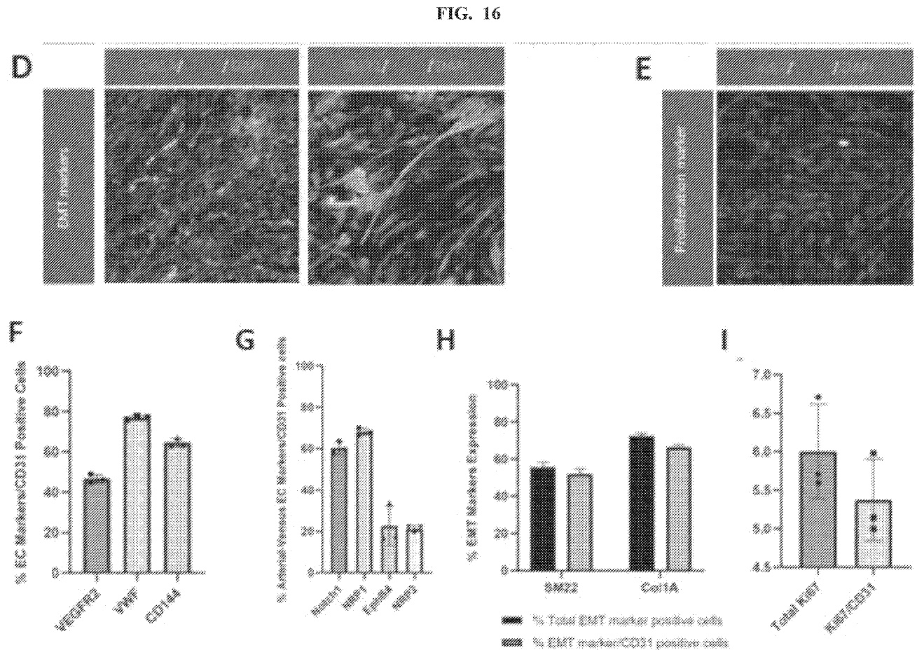

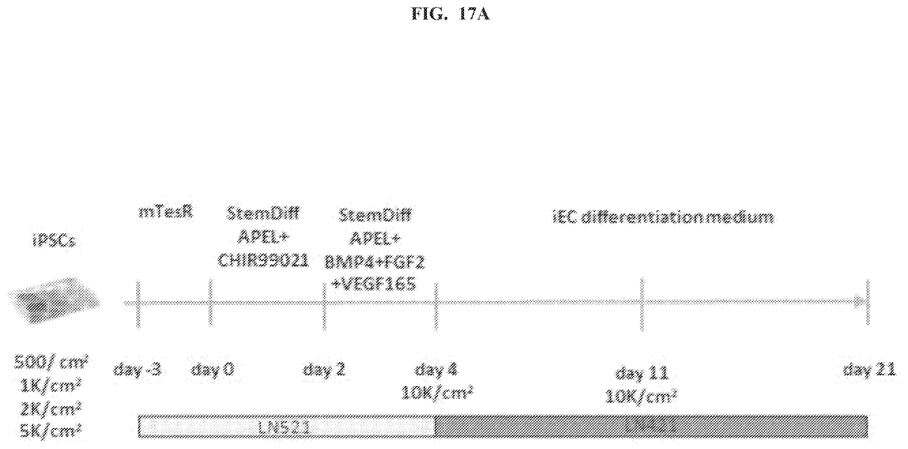

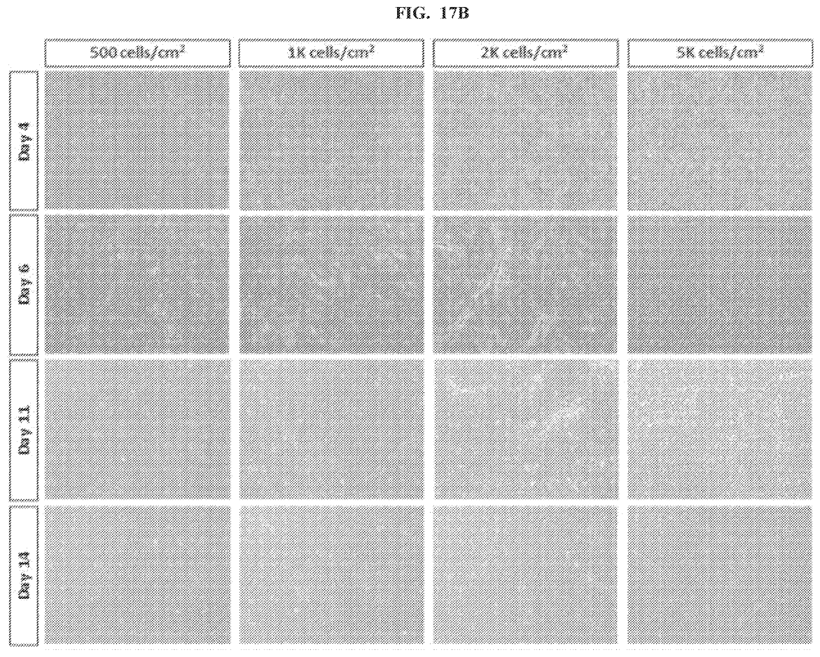

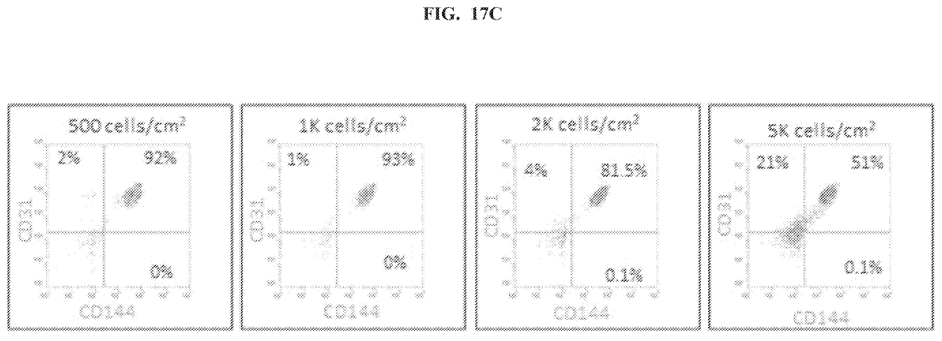

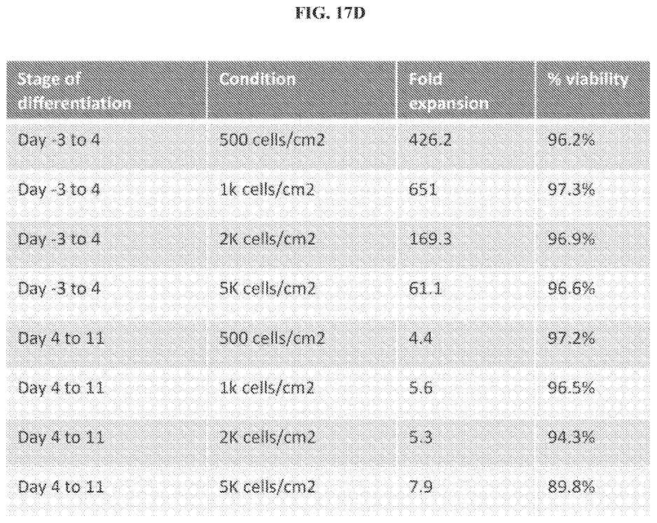

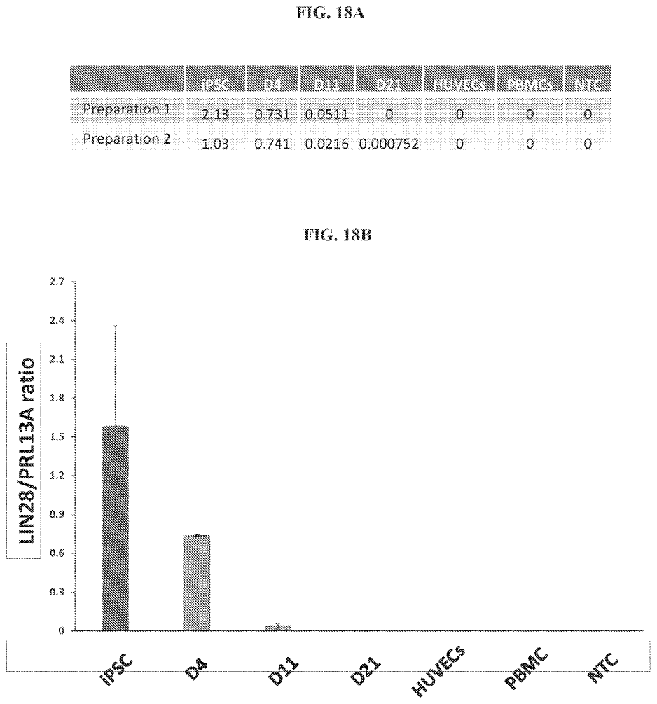

Perhaps of greater interest is the comparison of proteins that differentiate what may be considered ‘mature’ EC (HUVECS) from the stem-cell derived iECs, as these differences may reveal targets for further maturation or key functional differences between these two cell types. This comparison found 4,634 DEPs (FIG. 2A), with comparative pathway analysis on DEPs revealing multiple differential functional pathways between the cell types, including prominent upregulation of EMT and glycolysis in iEC (FIG. 2B) and concomitant upregulation of IFN-gamma (along with other inflammatory pathways) and Oxidative phosphorylation in HUVECs (FIG. 2C). Proteins with that were most upregulated in iECs included proteins related to nucleic acid metabolism (SAMHD1 and DDX58), TNF-alpha signaling via NF-κβ (DDX58 and SERPINB2), and IL-6 and IL-10 signaling (HMOX1). In HUVECs, proteins related to focal adhesions (FLT1 and COL4A2) and VEGF signaling (FLT1) were highly upregulated. Thus, gene set enrichment analysis identified pathways of interest in both iPSCs and endothelial cell types and demonstrated that iECs and primary HUVECs exhibit proteomic similarities important in maintaining endothelial cell function. Notably, the endothelial cell types contain similarities in key proteins and enrichment in pathways not present in iPSCs.

Screening of abundant Post Translational Modifications reveals differentially modified peptides across differentiation states. Total proteome can provide detailed insight into overall cell phenotype, but additional granularity into cell signaling and regulatory states may be gleaned from specific interrogation of PTMs between cells. While PTMs are typically difficult to detect without prior enrichment due to stoichiometric limitations, we were able to confidently identify several PTM sites across the cell lines (FIG. 3A). Of note, consistently more of the observed PTM sites were shared between HUVECs and iECs than between these cells individually and iPSCs and when shared between any two cell types, a notable proportion of sites demonstrated significantly altered abundance (FIG. 3A). To glean insight into functional implications of differential PTM findings, gene ontology analysis on proteins found in each PTM category were analyzed with DAVID Gene Ontology platform. For most PTMs, modification sites proteins related to cell adhesion and focal adhesion pathways were prevalent across the EC cell types, whereas PTMs on proteins related to chromatin and RNA binding.

As is true for most PTM profiling studies, inferring functional implications for observed PTMs is a significant challenge given limitations in databases that annotate functional impact of a large number of PTM sites. That being said, the granularity that PTM profiling can provide into cellular signaling state provides the opportunity to identify potential targets for further method development toward terminal iEC differentiation in vitro to mature phenotypes. For instance, we profiled the phosphorylation sites that demonstrated significant differences between the different cell types using the piNET platform to identify upstream kinases known to regulate observed phosphorylation sites. While many sites currently have no annotated regulatory kinase, we did observe several sites regulated by cyclin dependent kinase 1 (CDK1) whose phosphorylation was increased in iPSCs. This observation serves as a sanity check for these data, as CDK1 has a well-known role in maintaining pluripotency. A handful of other sites also had known regulatory kinases driving abundance changes between iECs and iPSCs, including two upregulated sites linked to HIPK2, a kinase which has previously been studied for a role in modulating endothelial cell differentiation and angiogenesis. Only two differential sites between iECs and HUVECs are linked to known regulatory kinases, implicating members of the PKC and CDK family of kinases in their differential phosphorylation. In addition to these general trends, a handful of particularly notable PTMs were observed. We observed a dual phosphorylation (S56) and lysine monomethylation (K64) site on the protein AKT2 that was significantly elevated in HUVECs vs iECs, despite the iECs expressing the largest total amount of AKT2 protein. Another potentially impactful PTM differences was observed on CTNNB1 (an effector of WNT that is present in transcription factor binding/signaling pathways), with lysine methylation at 354 that is only present in the endothelial cell types (FIG. 2C). Potential functional implications of these differential PTM sites are discussed in detail below. Overall, the PTM screening study corroborates our conclusions from total proteome analysis that the iECs are distinct from iPSCs in PTM profile, with more shared abundant PTMs with HUVECs than with their parental iPSCs.

In the present study we report a simple and reliable method to generate ECs from iPSCs in adherent cultures in a defined media utilizing small molecules and growth factors. This method is very efficient across multiple iPSCs lines derived from different donors giving rise to functional iECs with high efficiency within three weeks from iPSC stage. In order to arrive at this robust iEC differentiation method, various critical process parameter optimizations were performed that included optimization of starting iPSC colony size, seeding cell densities, culture media, timing of differentiation, cell harvesting and replating, and cell sorting. For example, initiating iEC differentiation with small iPSC colonies with chemical methods resulted in a consistent process with more reliable and efficient iEC differentiation compared to when colonies were larger than 200 μm (data not shown).

Specifically, in order to differentiate iPSC into iECs, activation of the canonical Wnt/β-catenin signaling pathway is a strategy widely used to give rise to mesodermal cells from embryonic state-like cells (Lindsley et al 2006, Zhao et al 2019), and vascular endothelial lineages are derived from mesoderm (Dyer & Patterson 2010). In this method, we activated the canonical Wnt signaling pathway through the inhibition of GSK3-mediated β-catenin phosphorylation (Wu & Pan 2010) using the small molecule CHIR99021. It is known that a short pulse active Wnt/β-catenin signaling from iPSC stage enhances expression of mesodermal genes by supporting an exit from pluripotency (Kreuser et al 2020). On the other hand, inhibiting the Wnt pathway leads to failure to generate Flk1⁺ (fetal liver kinase 1, or VEGFR2) mesodermal precursors and subsequent mature mesodermal lineages (Lindsley et al 2006). Once mesodermal specification is performed from pluripotency, use of basic FGF (FGF2) induces specification of the hemangioblast precursor of ECs, and the use of BMP4 is known to induce expression of Flk1/VEGFR2 (Dyer & Patterson 2010). At this stage, stem cell leukemia (SCL) and the adherens junction protein vascular endothelial (VE)-cadherin, markers of endothelial precursors, are also induced. Flk1/SCL hemangioblasts within the mesoderm form blood island clusters and are induced by vascular endothelial growth factor (VEGF)-A (Dyer & Patterson 2010), a critical morphogen known to drive specification of vascular endothelium (Oh et al 2021). In this study, we utilized these critical morphogens, namely FGF2, BMP4 and VEGF-A, to induce vascular endothelial cell progenitors from mesodermal cells. During this vascular mesoderm specification stage, a heterogenous population of cells are formed at this stage in the blood islands and undergo into different directions: the outer cells flatten and become ECs while the inner cells differentiate into hematopoietic cells (Dyer & Patterson 2010). We also observed two distinct populations at this stage of differentiation—peripheral cells at the edge of an iPSC colony that have a cobblestone-like morphology and cells in the center of the colonies that have a distinct morphology. The cobblestone-like cells lift easily from the plate and are re-plated to continue the vascular endothelium differentiation onto the next stages, when the endothelial-lineage cells continue to be matured with the additional of VEGF-A and other media growth supplements. At Day 11, iECs are dissociated and re-plated and further matured with the continued use of VEGF-A until final cell harvest. During human development, the ECs that will comprise the arteries are close to the notochord, which is a source of Sonic hedgehog source induces high levels of VEGF. On the other hand, to achieve a venous fate, lower Sonic hedgehog levels act to induce low levels of VEGF (Dyer & Patterson 2010). Our method gave rise to a mixed EC population, displaying arterial and venous lineage markers similar to other studies (Rufaihah et al 2013). Future studies may manipulate Sonic hedgehog pathway and concentrations of VEGF to direct the iECs into arterial or venous fate (Rosa et al 2019). The method reported here routinely results in a high percent of the iEC population (>80%) expressing CD31 and CD144 by Day 21 across multiple iPSC lines, which can be enriched >99% purity for iEC markers using MACS for CD31 cell surface protein. An early report also explored the differentiation of multiple iPSC lines into iECs and showed that iPSCs derived from skin fibroblasts and differentiated into iECs in 10 days presented a population expressing VEGFR2⁺, CD31⁺ and CD144⁺ in 1-5% of the cells; after sorting, iECs expressed more EC markers, formed network-like structures and had a cobblestone morphology (Taura et al 2009). Since protein expression is highly informative about cell state, phenotype and function of ECs and given the fact that there are currently no studies that have examined how the proteomic state of iPSC-derived ECs, we conducted an in-depth proteomic analysis using iECs derived from multiple donor iPSC lines. We believe that this information is critical for further development of iPSC-derived endothelial cells as they are being translated from the bench towards clinical regenerative cell therapy applications.

The proteomic comparison of iECs, iPSCs and HUVECs represents, to our knowledge, the most comprehensive if not the only of its kind to date. Principal component analysis demonstrated clearly that the proteomic state of iECs was more similar to HUVECs than iPSC, supporting our conclusions made from analysis of individual protein markers and RNA expression that this iPSC differentiation method was effective and efficient. This is quite significant given the known variability associated with iPSC-derived cell models that can be confounded by donor variability and genetic stability of iPSCs among various factors. Functional pathway analysis revealed several intriguing observations. For instance, proteins associated with spliceosomes were upregulated in iPSCs, agreeing with previous findings. As a splicing factor switch occurs during the differentiation of mesodermal cells to endothelial progenitor cells, further investigation may lead to an increased understanding of differentiation processes and mechanisms to control cell fate. In endothelial cell types, focal adhesions and regulation of actin cytoskeleton were upregulated. Focal adhesions play an important role in endothelial cell morphology and function, anchoring cells to the vascular wall and aiding in vascular responses to physical and chemical stimuli. Taken together, we concluded that the proteomic differences between iPSC and ECs were consistent with their highly disparate functional states. We then turned to the functional pathways that differentiate proteomic states of iECs from HUVECs, as these pathways may provide targets for further maturation of iECs. Interestingly, proteomic data implied that while mitochondrial metabolism/oxidative phosphorylation was upregulated in HUVEC, the iEC expressed proteins more consistent with a dominant glycolyltic state. Together these data indicate that there may be key differences in metabolic function between nascent iECs and perhaps more ‘native’ primary tissue-derived ECs and that interventions to promote mitochondrial function and oxidative metabolism may assist in further development of iECs. Total protein data implied upregulation of pro-inflammatory, interferon signaling in HUVECs relative to more naïve iECs. The strong interferon signaling signature that was enriched in HUVECs may reflect their prior development within an intact human system, where ECs participate in immune surveillance and response It will be of interest to explore in future whether exposure to immune-related inflammatory signals is a key component of EC maturation or an auxiliary finding.

Post translational modification is a major mode for regulating protein-protein interactions and enzyme activity. We were able to detect hundreds of PTMs across our proteins without a priori chemical enrichment. From our survey, PTMs on proteins related to many pathways intrinsic to iPSC and EC function were identified with a handful demonstrating interesting abundance patterns that may implicate regulation of important EC differentiation pathways. Among these, we highlighted and confirmed from the raw data a dual methylation and phosphorylation on AKT2 (phosphorylated serine 56 and monomethylated lysine 64) as well as dimethylation of lysine 374 on β-catenin. AKT2 is a major regulator of metabolic pathways, whereas β-catenin is critical mediator of Wnt signaling. Since metabolic-related proteomic differences were prominent between HUVECs and iECs and, as discussed above, Wnt signaling is critical for the efficient differentiation of iECs from iPSCs, these two PTM sites were highlighted for in depth consideration. Little is known regarding the functional implications of the PTM sites quantified on Akt2. Neither of these residues have existing annotation for these PTMs, though the methylation site may impact a known ubiquitinylation site and thus impact protein stability. AKT2 is involved in metabolic regulation, and cell cycle progression, thus this differential PTM could reflect altered AKT2 activity pertinent to functional iECs and HUVECs differences. Future studies manipulating these specific residues on AKT2 in iECs and HUVECs will test whether these dual PTMs are functionally relevant to EC differentiation and maturation.

The dimethylation site identified on β-catenin is supported by minimally more functional data in the literature. Degradation of nuclear β-catenin depends on Kdm2a-induced demethylation, which presumably promotes reduced transcriptional activity of this end-effector of the Wnt signaling pathway. Our observation of increased dimethylation of K374 coupled with reduced expression of KDM2a and b in endothelial cell types indicates that enhanced methylation may promote nuclear stabilization of β-catenin and promote efficient differentiation of iECs. This information can be used to further explore the role of Wnt signaling in iEC differentiation and maintenance of EC function and highlights either the demethylases KDM2A and B, or the as yet unknown lysine methyl transferase acting on β-catenin as potential molecular targets to improve iEC differentiation efficiency in future studies.

In summary, we report a robust, improved and highly efficient method for the differentiation of iEC across multiple donor human iPSC lines, which provides excellent promise as a cellular therapeutic development for vascular applications. We have extensively characterized both the total and abundant PTM proteomes of the iECs produced and shown that while the iEC are highly similar to a primary reference ECs (HUVECs).

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of generating endothelial cells from induced pluripotent stem cells (iECs), comprising
(i) differentiating induced pluripotent stem cells (iPSCs) into mesoderm cells by:
removing cGMP feeder-free iPSC maintenance medium from plated induced pluripotent stem cells (iPSCs) and adding mesoderm induction medium, and
culturing the iPSCs for about 1.5-2.5 days in about 36-38 C and in the presence of about 4-6% CO₂ to generate mesoderm cells;
(ii) differentiating the mesoderm cells into vascular progenitor cells by:
removing the mesoderm induction medium and adding vascular progenitor medium, and
culturing the cells for about 1.5-2.5 days in about 36-38 C and in the presence of about 4-6% CO₂ to generate vascular progenitor cells;
(iii) differentiating the vascular progenitor cells into iECs by:
dissociating the vascular progenitor cells by using a first dissociation enzyme for about 4-6 minutes at 36-38 C and in the presence of about 4-6% CO₂, wherein the dissociation enzyme comprises an animal origin free enzyme that cleaves peptide bonds on the C-terminal sides of lysine and arginine;
neutralizing the vascular progenitor cells by using basal endothelial cell growth medium MV2 plus about 9-11% human AB serum, spinning the vascular progenitor cell solution, removing the basal endothelial medium, and resuspending the vascular progenitor cells in GMP-grade iEC differentiation medium, wherein the human AB serum comprises human serum lacking antibodies against A and B blood-type antigens that was converted from Pooled Plasma (Human), Solvent/Detergent Treated;
plating the vascular progenitor cells at about 9,000-11,000 cells/cm² with iEC differentiation medium onto a dish that was previously coated with 4-6 ug/ml human recombinant laminin 421 in PBS Mg+/Ca+ for about 1.5-2.5 hours at about 36-38 C and in the presence of about 4-6% CO₂,
culturing the vascular progenitor cells at about 36-38 C and in the presence of about 4-6% CO₂ for about 6-8 days, feeding about every other day with iEC differentiation medium to generate iECs; and
(iv) further culturing the iECs by:
dissociating the iECs by using animal origin free enzyme that cleaves peptide bonds on the C-terminal sides of lysine and arginine (1×) for about 4-6 minutes at about 36-38 C and in the presence of about 4-6% CO₂;
neutralizing the iECs using by using basal endothelial cell growth medium MV2 plus 9-11% Human AB serum, spinning the iEC solution, removing the basal endothelial medium, and resuspending the iECs in GMP-grade iEC differentiation medium, wherein the human AB serum comprises human serum lacking antibodies against A and B blood-type antigens that was converted from Pooled Plasma (Human), Solvent/Detergent Treated;
plating the iECs at about 9,000-11,000 cells/cm² with iEC differentiation medium onto a dish that was previously coated with Sug/ml human recombinant laminin 421 in PBS Mg+/Ca+ for about 1.5-2.5 hours at about 36-38 C and in the presence of about 4-6% CO₂,
culturing the iECs at about 36-38 C and in the presence of about 4-6% CO₂ for about 9-11 days, feeding about every other day with iEC medium, thereby generating iECs expressing CD31 and CD144.

2. The method of claim 1, further comprising dissociating the iECs that are generated in step (iv).

3. The method of claim 1, further comprising providing plated iPSCs, wherein the plated iPSCs are made by:
dissociating iPSCs with a cGMP enzyme-free human pluripotent stem cell selection and passaging reagent for 5 minutes and neutralizing the iPSCs with iPSC maintenance medium;
triturating the iPSCs about 10-15 times to break large colonies of iPSCs to smaller colonies of iPSCs and to have a homogenous cell population that is about 60-200 um diameter per iPSC colony;
dissociating a subset of colony solution as single cells by using a dissociation solution, for 5 minutes at 37 C water bath, wherein the dissociation solution comprises a cell detachment solution of proteolytic and collagenolytic enzymes and does not contain mammalian or bacterial derived products;
neutralizing the dissociation solution by using a cGMP feeder-free iPSC maintenance medium, spinning the cell solution, removing the medium and resuspending the iPSCs in iPSC maintenance medium;
removing laminin solution from a plating dish that previously coated with Sug/ml human recombinant laminin 521 in PBS Mg+/Ca+ for 2 hours in 37 C and in the presence of 5% CO₂;
adding cGMP feeder-free iPSC maintenance medium to the plating dish;
adding about 2000 iPSCs/cm² to the plating dish and culturing at 37 C and in the presence of 5% CO₂; and
changing the cGMP feeder-free iPSC maintenance medium daily for an additional 2 days.

4. The method of claim 1, wherein the mesoderm induction medium comprises a final concentration of about 5-7 uM CHIR99021 and about 1:900-1:1100 Antibiotic-Antimycotic (PSA) in iPSC differentiation medium, wherein the iPSC differentiation medium comprises a defined, animal component-free medium for differentiation of human ES and iPS cells to multiple lineages.

5. The method of claim 1, wherein the vascular progenitor medium comprises final concentrations of about 20-30 ng/ml Bone Morphogenetic Protein 4 (BMP4), about 8-12 ng/ml FGF2, about 40-60 ng/ml VEGF165 and about 1:900-1:1100 Antibiotic-Antimycotic (PSA) in iPSC differentiation medium.

6. The method of claim 1, wherein the iEC differentiation medium comprises Basal MV2, PSA, Human AB Serum (AB serum), Epidermal Growth Factor (EGF), Basic Fibroblast Growth Factor (FGF2), Long R3 Insulin-like Growth Factor (IGF-1), Vascular Endothelial Growth Factor 165 (VEGF 165), Ascorbic acid, and Hydrocortisone.

7. The method of claim 1, wherein the iEC differentiation medium comprises Basal MV2, 1:900-1:1100 PSA, 0.05-0.15 ml/ml Human AB Serum (AB serum), 4-6 ng/ml Epidermal Growth Factor (EGF), 8-12 ng/ml Basic Fibroblast Growth Factor (FGF2), 18-22 ng/ml Long R3 Insulin-like Growth Factor (IGF-1), 240-280 ng/ml Vascular Endothelial Growth Factor 165 (VEGF 165), 0.5-1.5 ug/ml Ascorbic acid, and 0.1-0.3 ug/ml Hydrocortisone.

8. The method of claim 1, comprising
(i) differentiating induced pluripotent stem cells (iPSCs) into mesoderm cells by:
removing cGMP feeder-free iPSC maintenance medium from plated induced pluripotent stem cells (iPSCs) and adding mesoderm induction medium, and
culturing the iPSCs for about 2 days in about 37 C and in the presence of about 5% CO₂ to generate mesoderm cells;
(ii) differentiating the mesoderm cells into vascular progenitor cells by:
removing the mesoderm induction medium and adding vascular progenitor medium, and
culturing the cells for about 2 days in about 37 C and in the presence of about 5% CO₂ to generate vascular progenitor cells;
(iii) differentiating the vascular progenitor cells into iECs by:
dissociating the vascular progenitor cells by using a first dissociation enzyme for about 5 minutes at 37 C and in the presence of about 5% CO₂, wherein the dissociation enzyme comprises an animal origin free enzyme that cleaves peptide bonds on the C-terminal sides of lysine and arginine;
neutralizing the vascular progenitor cells by using basal endothelial cell growth medium MV2 plus about 10% human AB serum, spinning the vascular progenitor cell solution, removing the basal endothelial medium, and resuspending the vascular progenitor cells in GMP-grade iEC differentiation medium, wherein the human AB serum comprises human serum lacking antibodies against A and B blood-type antigens that was converted from Pooled Plasma (Human), Solvent/Detergent Treated;
plating the vascular progenitor cells at about 10,000 cells/cm² with iEC differentiation medium onto a dish that was previously coated with Sug/ml human recombinant laminin 421 in PBS Mg+/Ca+ for about 2 hours at about 37 C and in the presence of about 5% CO₂,
culturing the vascular progenitor cells at about 37 C and in the presence of about 5% CO₂ for about 7 days, feeding about every other day with iEC differentiation medium to generate iECs; and
(iv) further culturing the iECs by:
dissociating the iECs by using animal origin free enzyme that cleaves peptide bonds on the C-terminal sides of lysine and arginine (1×) for about 5 minutes at about 37 C and in the presence of about 5% CO₂;
neutralizing the iECs using by using basal endothelial cell growth medium MV2 plus 10% Human AB serum, spinning the iEC solution, removing the basal endothelial medium, and resuspending the iECs in GMP-grade iEC differentiation medium, wherein the human AB serum comprises human serum lacking antibodies against A and B blood-type antigens that was converted from Pooled Plasma (Human), Solvent/Detergent Treated;
plating the iECs at about 10,000 cells/cm² with iEC differentiation medium onto a dish that was previously coated with Sug/ml human recombinant laminin 421 in PBS Mg+/Ca+ for about 2 hours at about 37 C and in the presence of about 5% CO₂,
culturing the iECs at about 37 C and in the presence of about 5% CO₂ for about 10 days, feeding about every other day with iEC medium, thereby generating iECs expressing CD31 and CD144.

9. The method of claim 1, further comprising dissociating the iECs that are generated in step (iv).

10. The method of claim 1, further comprising providing plated iPSCs, wherein the plated iPSCs are made by:
dissociating iPSCs with a cGMP enzyme-free human pluripotent stem cell selection and passaging reagent for 5 minutes and neutralizing the iPSCs with iPSC maintenance medium;
triturating the iPSCs about 10-15 times to break large colonies of iPSCs to smaller colonies of iPSCs and to have a homogenous cell population that is about 60-200 um diameter per iPSC colony;
dissociating a subset of colony solution as single cells by using a dissociation solution, for 5 minutes at 37 C water bath, wherein the dissociation solution comprises a cell detachment solution of proteolytic and collagenolytic enzymes and does not contain mammalian or bacterial derived products;
neutralizing the dissociation solution by using a cGMP feeder-free iPSC maintenance medium, spinning the cell solution, removing the medium and resuspending the iPSCs in iPSC maintenance medium;
removing laminin solution from a plating dish that previously coated with Sug/ml human recombinant laminin 521 in PBS Mg+/Ca+ for 2 hours in 37 C and in the presence of 5% CO₂;
adding cGMP feeder-free iPSC maintenance medium to the plating dish;
adding about 2000 iPSCs/cm² to the plating dish and culturing at 37 C and in the presence of 5% CO₂; and
changing the cGMP feeder-free iPSC maintenance medium daily for an additional 2 days.

11. The method of claim 1, wherein the mesoderm induction medium comprises a final concentration of about 6 uM CHIR99021 and about 1:1000 Antibiotic-Antimycotic (PSA) in iPSC differentiation medium, wherein the iPSC differentiation medium comprises a defined, animal component-free medium for differentiation of human ES and iPS cells to multiple lineages.

12. The method of claim 1, wherein the vascular progenitor medium comprises final concentrations of about 25 ng/ml Bone Morphogenetic Protein 4 (BMP4), about 10 ng/ml FGF2, about 50 ng/ml VEGF165 and about 1:1000 Antibiotic-Antimycotic (PSA) in iPSC differentiation medium.

13. The method of claim 1, wherein the iEC differentiation medium comprises Basal MV2, PSA, Human AB Serum (AB serum), Epidermal Growth Factor (EGF), Basic Fibroblast Growth Factor (FGF2), Long R3 Insulin-like Growth Factor (IGF-1), Vascular Endothelial Growth Factor 165 (VEGF 165), Ascorbic acid, and Hydrocortisone.

14. The method of claim 1, wherein the iEC differentiation medium comprises Basal MV2, 1:1000 PSA, 0.1 ml/ml Human AB Serum (AB serum), 5 ng/ml Epidermal Growth Factor (EGF), 10 ng/ml Basic Fibroblast Growth Factor (FGF2), 20 ng/ml Long R3 Insulin-like Growth Factor (IGF-1), 260 ng/ml Vascular Endothelial Growth Factor 165 (VEGF 165), 1 ug/ml Ascorbic acid, and 0.2 ug/ml Hydrocortisone.

15. A quantity of iECs made by the method of claim 1.

16. The quantity of iECs of claim 15, wherein the quantity is a clinically meaningful quantity.

17. A method of treating a condition in a subject in need thereof, comprising
administering iECs made by the method of claim 1 to the subject in need thereof.

18. The method of claim 17, wherein the condition comprises transplantation, tissue reconstruction, ischemic trauma or injury, diabetes, cancer, or cosmetic condition.

19. The method of claim 17, wherein treating a condition comprises treating a wound, treating an amputation, treating a damaged organ, or treating necrosed tissue.

20. A method of drug screening, comprising:
contacting a test drug to a population of iECs made by the method claim 1;
measuring one or more parameters of the iECs; and
identifying the drug as a candidate based on the measured one or more parameters.
